(19) United States
(12) Patent Application Publication
(10) Pub. No.: US 2024/0409577 A1
(43) Pub. Date: Dec. 12, 2024

(54) FORMATION OF AMIDINES AND INTRAMOLECULAR REACTION THEREOF

(71) Applicants: Brett VanVeller, Ames, IA (US); Emily O'Brien, Cedar Rapids, IA (US); Krishna K. Sharma, Ames, IA (US); Jacob Byerly-Duke, Ames, IA (US); Brendan J. Wall, Bloomington, IL (US); Rania Ibrahim, Ames, IA (US)

(72) Inventors: Brett VanVeller, Ames, IA (US); Emily O'Brien, Cedar Rapids, IA (US); Krishna K. Sharma, Ames, IA (US); Jacob Byerly-Duke, Ames, IA (US); Brendan J. Wall, Bloomington, IL (US); Rania Ibrahim, Ames, IA (US)

(21) Appl. No.: 18/812,489

(22) Filed: Aug. 22, 2024

Related U.S. Application Data
(63) Continuation-in-part of application No. 18/598,811, filed on Mar. 7, 2024.
(60) Provisional application No. 63/488,879, filed on Mar. 7, 2023.

(51) Int. Cl. C07K 1/04

(52) U.S. Cl. CPC C07K 1/04

(57) ABSTRACT

A method of conversion of imines substituted with —X—R¹ into amidines includes reacting a starting material compound including an imine that comprises —X—R¹ bound to an imine carbon atom of the imine with a nitrogen-containing reagent to form a product compound including an amidine in place of the imine —X—R¹ in the starting material compound. The nitrogen-containing reagent includes a primary amine, a secondary amine, an ammonium salt, or a combination thereof. The variable X is —S—, —O—, or —NH—. The variable R¹ is substituted or unsubstituted (C₁-C₂₀) hydrocarbyl, wherein R¹ is optionally bonded to a solid support. A method of forming a cyclized product includes intramolecularly reacting an amidine with a carbonyl carbon to form the cyclized product including a 4H-imidazolone and/or a derivative thereof.

### CROSS-REFERENCE TO RELATED APPLICATIONS

This application is a continuation-in-part of and claims the benefit of priority under 35 U.S.C. § 120 to U.S. Utility application Ser. No. 18/598,811 filed Mar. 7, 2024, which claims the benefit under 35 U.S.C. § 119 (e) of U.S. Provisional Patent Application Ser. No. 63/488,879 filed Mar. 7, 2023, the disclosures of which are incorporated herein in their entirety by reference.

### STATEMENT OF GOVERNMENT SUPPORT

This invention was made with Government support under R35 GM142883 awarded by the National Institutes of Health. The Government has certain rights in the invention.

### BACKGROUND

Numerous isosteres have been developed to mimic the shape and function of the native amide bond. One isostere in particular—the amidine—can occur naturally, but also contrasts with amides by displaying a dynamic hydrogen-bonding motif (FIG. 1A). This unique ability of amidines to alter their hydrogen-bond donating and accepting character was exploited by Boger and coworkers to counter the resistance of bacteria towards vancomycin (1). Likewise, amidines are key features of other therapeutic enzyme/receptor inhibitors such as the anti-psychotic drug Clozapine, anti-infectives like Pafuramidine and Pentamidine, and the FDA approved anticoagulant Dabigatran. Further, beyond the ability of amidines to operate as hydrogen-bond shape shifters, the additional valence compared to the amide furnishes another site to append substituents (R, FIG. 1A).

While these distinct properties of amidines have served as design elements in numerous pharmaceutical compounds, polymeric materials, and even prebiotic building blocks, amidines have received relatively little attention in peptides due to a lack of compatibility and general methods for incorporation into peptides using standard Fmoc solid-phase peptide synthesis (SPPS) techniques.

Recent syntheses of amidines in peptidic molecules have exploited thioamides as a site that can be activated with Ag(I) salts for conversion into amidines via reaction with nucleophiles. The installation of thioamides into peptides using standard SPPS procedures can be achieved with activated thioacyl amino acid precursors derived from commercially available Fmoc-amino acids. Thus, in principle, a thioamide-containing peptide should provide an avenue for the site-selective insertion of amidines into peptides. Unfortunately, examples of such chemistry within the context of polypeptides are noticeably scarce. The paucity of literature around the Ag(I)-promoted conversion of thioamides into amidines along the peptide backbone is likely due to a rapid intramolecular 5-exo-trig attack from an adjacent backbone amide onto the thioamide carbonyl (3→4, FIG. 1B), crippling any chance for intermolecular attack by an amine nucleophile.

This type of ring closure has been observed previously for macrocycles in which both the N-terminal and/or C-terminal amides that bracket the thioamide along the backbone can cyclize, as well as for other nucleophiles that are in close proximity to the thioamide. These results likely explain why this seemingly facile method to access amidines has not been successful for linear peptides. Indeed, to our knowledge, only two examples of Ag(I)-mediated installation of amidines into peptide-like molecules have been reported (FIGS. 1A, 1 and 2). The first was Boger's amidine-containing vancomycin synthesis mentioned above (1), and in the second, Yudin and coworkers demonstrated the conversion of a thioamide into an amidine within a small macrocyclic peptide (2). In both of these examples, the constrained nature of the macrocycles likely plays a role in preventing the 5-exo-trig cyclization, as formation of a planar oxazole within each small ring is conformationally disfavored.

Incorporation of heterocyclic motifs along the peptide backbone represents a critical tool to address issues in peptide drug metabolism and cell permeability. Nature has evolved its own biological machinery to include heterocycles and expand the complexity of peptides beyond the standard suite of canonical amino acids. The late biochemist Christopher T. Walsh credited these heterocycles as “a recurring motif in Nature's medicinal chemistry toolbox”. In nature, backbone heterocycles are installed via cyclization from an adjacent side-chain onto the backbone amide linkage, leading to oxazole-type (from Ser or Thr), and thiazole-type (from Cys) heterocycles (FIG. 6A left). Synthetic chemists have mimicked this approach using on-resin chemical activation of the amide bond or by incorporating non-natural side-chains into linear peptides to access aromatic backbone heterocycles such as oxazoles, thiazoles, imidazoles, pyrazoles, oxadiazoles, 4-imidazolidinones, 2-imidazolidines, 1,2,4-triazoles, 1,2,3-triazoles, and iminohydantoins. The substitution pattern imparted by all of these heterocycles generates a 1,3-trans-amide-like conformation along the peptide backbone (FIG. 6A left) which mimics the native trans-amide conformation.

One can imagine a second option for backbone heterocyclic installation, derived solely from the amide linkage. These heterocycles would yield a 1,2-cis-amide-like motif along the peptide backbone (FIG. 6A right), geometrically constraining the amide bond to a non-native cis-amide conformation. Because the amide bond of peptides only exists in the cis-conformation 0.1-0.2% of the time (at room temperature), cis-amide bond surrogates are of exceeding interest to synthetic and medicinal chemists for their ability to initiate turn motifs in peptides, reduce proteolysis, and facilitate peptide macrocyclization.

Current methods to insert heterocycles which lock the cis-amide conformation, however, are not ‘plug-and-play’ and suffer from significant challenges regarding implementation and compatibility with solid-phase peptide synthesis (SPPS)-the work-horse method for peptide synthesis. Previous methods have focused exclusively on cis-substituted triazoles and tetrazoles. Many of the reported methods are not compatible or provide unreliable results on solid phase, instead requiring cumbersome pre-synthesis of the heterocyclic precursors prior to installation into tripeptide fragments ahead of coupling to solid support.

### SUMMARY OF THE INVENTION

Various aspects of the present invention provide a method that includes reacting a starting material compound including an imine that includes —X—R¹ bound to an imine carbon atom of the imine with a nitrogen-containing reagent to form a product compound including an amidine in place of the imine —X—R¹ in the starting material compound. The nitrogen-containing reagent includes a primary amine, a secondary amine, an ammonium salt, or a combination thereof. The variable X is —S—, —O—, or —NH—. The variable R¹ is substituted or unsubstituted (C₁-C₂₀) hydrocarbyl, wherein R¹ is optionally bonded to a solid support

Various aspects of the present invention provide a method including reacting a starting material compound including an imine that includes —X—R¹ bound to an imine carbon atom of the imine, wherein the starting material compound or the nitrogen-containing reagent is bonded to a solid support, with a nitrogen-containing reagent in a reaction milieu including a polar aprotic solvent and an optional fluoroalcohol to form a product compound including an amidine in place of the imine —X—R¹ in the starting material compound. The nitrogen-containing reagent includes a primary amine, a secondary amine, an ammonium salt, or a combination thereof. The variable X is —S—, —O—, or —NH—. The variable R¹ is substituted or unsubstituted (C₁-C₂₀) hydrocarbyl, wherein R¹ is optionally bonded to the solid support.

X-Amides can be converted into imine —X—R¹ during SPPS to preserve the α-C stereochemistry thereof during subsequent elongation of the peptide on solid support; for example, thioamides can be converted into thioimidates (5, FIG. 1C) during SPPS to preserve the α-C stereochemistry of thioamides during subsequent elongation of the peptide on solid support. The basic conditions of SPPS generally maintain the thioimidate in its deprotonated state. The basicity of the thioimidate, however, means that it can be rendered more electrophilic upon protonation (5⁺). In various methods of the present invention, amidines can be introduced into peptides through the imine —X—R¹ rather than the X-amide; for example, amidines can be introduced into peptides through the thioimidate rather than the thioamide (5→6, FIG. 1C). In addition to being the first general approach to incorporate amidines into peptide backbones, this approach has several desirable features. For example, various aspects of the method of the present invention can avoid the use of precious transition metals such as silver. In various aspects of the method of the present invention, the amidine or the nitrogen-containing reagent can be introduced on-resin prior to cleavage and deprotection of the peptide. The ability to introduce amidines or nitrogen-containing reagents while on resin can increase the generality of the method; for example, competing nucleophiles such as the reactive side-chain functional groups along the peptide can be capped. Due to reactivity that renders existing methods unsuitable for the installation of amidines along the main chain of peptides, an X-amide can be protected as imine —X—R¹; another advantage of various embodiments of the method of the present invention is that it can be combined with an imine —X—R¹ protection protocol for X-amides, such as during SPPS, wherein the imine —X—R¹ can then be exploited for installation of the amidine, providing enhanced utility and efficiency to the protection protocol. Various embodiments of the present method provide a synthetic path to access unexplored peptide designs and architectures for new therapeutics made possible by the unique properties of amidines.

Various aspects of the method of forming the product compound including the amidine further includes intramolecularly reacting the amidine in the product compound with a carbonyl carbon in the product compound to form a cyclized product including a 4H-imidazolone and/or a derivative thereof.

Various aspects of the present invention provide a method of forming a cyclized product. The method includes intramolecularly reacting a compound including the structure:

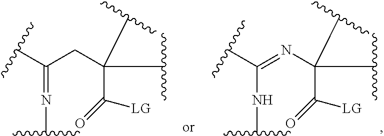

to form a cyclized product including the structure:

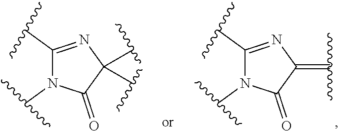 [SMILES: *C1=NC(*)(*)C(=O)N1* ; *C1=NC(=*)C(=O)N1*]

wherein LG is a leaving group.

In various aspects, the method of forming the cyclized product via intramolecular reaction of the amidine provides various advantages. For example, the method can provide convenient access to a 4H-imidazolone ring. In various aspects, the method can provide access to the imidazolone ring from starting materials that were previously unknown to be precursors for imidazolones. In various aspects, the method can provide access to the imidazolone ring or at higher yield, greater efficiency, less toxic materials, or a combination thereof, as compared to other methods for accessing imidazolone rings in the same or similar molecules. In various aspects of the present invention, the amide in the imidazolone ring can be locked in a cis-amide conformation; as such, the method can provide convenient access to a less thermodynamically favored conformation of the amide. In various aspects, the cyclized product can be conveniently formed in-situ after formation of the amidine-containing compound (e.g., the product compound including the amidine), providing simple and efficient access to the cyclized product.

Imidazolones represent an important class of heterocycles present in a wide-range of pharmaceuticals, intermediates of biologically relevant scaffolds, reactive handles for further functionalization, metabolites, bioactive natural products, and serve as the active chromophore in green fluorescent protein (GFP). Recently, imidazolones have received attention for their ability to act as a non-aromatic amide bond bioisotere which improves pharmacological properties. Various aspects of the present method provide a tandem amidine installation and cyclization with an adjacent ester to yield (4H)-imidazolone products. Various aspects of the present method can use amino acid building blocks to access the first examples of α-chiral imidazolones which have been previously inaccessible. Various aspects of the present method can be amenable to on-resin installation (e.g., with the starting material compound or the nitrogen-containing reagent bound to the solid support) which can be seamlessly integrated into existing solid-phase peptide synthesis (SPPS) protocols. Various aspects of the present method can provide peptide imidazolones that are potent cis-amide bond surrogates which pre-organize linear peptides for head-to-tail macrocyclization and cyclization of peptides. Various aspects of the present method represent the first general and widely applicable approach to backbone and side-chain insertion of imidazolone bioisosteres at various positions in linear and cyclic peptides.

### BRIEF DESCRIPTION OF THE FIGURES

The drawings illustrate generally, by way of example, but not by way of limitation, various aspects of the present invention.

FIG. 1A illustrates a scheme showing the malleable hydrogen-bonding motif of amidines, as well as structures of conformationally constrained peptidic molecules that allow for application of an Ag(I)-promoted conversion of thioamide into amidine.

FIG. 1B illustrates an Ag(I)-promoted method to convert thioamides into amidines, which is not applicable to linear peptides because of competing intramolecular attack by adjacent amides.

FIG. 1C illustrates a method for amidine insertion into linear peptides, in accordance with various aspects.

FIG. 2A illustrates the synthesis of N-phenylethyl-4-(pyren-4-yl) butanethioamide, in accordance with various aspects.

FIG. 2B illustrates the synthesis of N-phenylethyl-4-(pyren-4-yl) butanethioimidate (7), in accordance with various aspects.

FIG. 2C illustrates the conversion of a thioimidate into an amidine, in accordance with various aspects.

FIG. 2D illustrates the conversion of a thioimidate into an amidine in the presence of a fluoroalcohol, in accordance with various aspects.

FIG. 3A illustrates the synthesis of Boc-Phe(S)-Ala-OMe dipeptide, in accordance with various aspects.

FIG. 3B illustrates the synthesis of Boc-Gly-Phe(S)-Ala-OMe tripeptide (3), in accordance with various aspects.

FIG. 3C illustrates cleavage of peptides from solid support, in accordance with various aspects.

FIG. 4A illustrates the conversion of a thioimidate into an amidine in a peptide and on solid support, wherein R′=1-pyrenebutyric acid, in accordance with various aspects.

FIG. 4B illustrates absorbance versus retention time for a peptide trimer prepared for D-phe versus a peptide trimer prepared from L-Phe, in accordance with various aspects.

FIG. 4C illustrates formation of oxazole and related side-products during amidine formation from thioamides using a silver-containing catalyst.

FIG. 5 illustrates biologically relevant amidine-containing peptides formed via conversion of thioimidate into amidine, in accordance with various aspects.

FIG. 6A illustrates a diagram demonstrating that heterocycles can geometrically restrict a peptide backbone to either cis-amide-like or trans-amide-like motifs, in accordance with various aspects.

FIG. 6B illustrates a diagram demonstrating that (4H)-imidazolones can act as non-aromatic amide bond isosteres.

FIG. 6C illustrates a diagram illustration production of imidazolones via amidine cyclization, in accordance with various aspects.

FIG. 7 illustrates various examples of (4H)-imidazolones present in pharmaceuticals, herbicides, or natural products.

FIG. 8 illustrates a chemical reaction of a thioimidate to an imidazolone, in accordance with various aspects.

FIG. 9 illustrates scope of various amine nucleophiles tolerated in imidazolone formation, in accordance with various aspects.

FIG. 10 illustrates a one-pot imidazolone cyclization-aldol condensation to access benzylidene imidazolones which mimic GFP hexapeptide, in accordance with various aspects.

FIG. 11 illustrates stereoretentive imidazolone formation with primary amine nucleophiles in accordance with various aspects.

FIG. 12 illustrates a chemical reaction of a thioimidate to an imidazolone, in accordance with various aspects.

FIG. 13 illustrates on-resin N-terminal imidazolone formation on Apidaecin Ib (1-7).

FIG. 14 illustrates imidazolone cyclization on Rink amide resin linker and subsequent elongation toward C-terminal imidazolone TRH analogue (28), in accordance with various aspects.

FIG. 15 illustrates imidazolone formation on Lys side-chain to yield AGE-related modification (29a) and subsequent elongation to the Lys branched peptide (29b), in accordance with various aspects.

FIG. 16 illustrates modified installation conditions enabling insertion of α-chiral imidazolones with low epimerization, in accordance with various aspects.

FIG. 17 illustrates the use of imidazolones as cis-amide surrogates to pre-organize head-to-tail macrocyclization of Mahafacyclin B analogue with bioisosteric replacement, in accordance with various aspects.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR) R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “organic group” as used herein refers to any carbon-containing functional group. Examples can include an oxygen-containing group such as an alkoxy group, aryloxy group, aralkyloxy group, oxo (carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀) hydrocarbyl, wherein R can be hydrogen (in examples that include other carbon atoms) or a carbon-based moiety, and wherein the carbon-based moiety can be substituted or unsubstituted.

The term “alkyl” as used herein refers to straight chain and branched alkyl groups and cycloalkyl groups having from 1 to 40 carbon atoms, 1 to about 20 carbon atoms, 1 to 12 carbons or, in some aspects, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain aspects there is no hydrocarbyl group. A hydrocarbylene group is a diradical hydrocarbon, e.g., a hydrocarbon that is bonded at two locations.

### Method for Conversion of an Imine —X—R¹ to an Amidine

Various aspects of the present invention provide a method that includes reacting a starting material compound including an imine that includes —X—R¹ bound to an imine carbon atom of the imine with a nitrogen-containing reagent to form a product compound including an amidine in place of the imine —X—R¹ in the starting material compound. The nitrogen-containing reagent includes a primary amine, a secondary amine, an ammonium salt, or a combination thereof. The variable X is —S—, —O—, or —NH—.

The imine —X—R¹ can be directly bonded to two carbon atoms in the starting material compound (i.e., via a bond from the imine carbon atom to another carbon atom in the starting material compound, and via a bond from the imine nitrogen atom to a different carbon atom in the starting material compound than the carbon atom that is bonded to the imine carbon atom), such as to two carbon atoms of a peptide backbone such that the imine —X—R¹ is part of the peptide backbone. As used herein, the term “imine carbon atom” refers to the carbon atom of the imine that has two bonds to nitrogen. The variable R¹ can be substituted or unsubstituted (C₁-C₂₀)hydrocarbyl optionally bonded to a solid support, such as (C₁-C₂₀)alkyl or methyl. In various aspects, R¹ is bonded to the solid support. In various aspects, R¹ is free of bonds to a solid support.

If the variable X is —S—, then the imine —S—R¹ is a thioimidate. The thioimidate includes —S—R¹, wherein R¹ is substituted or unsubstituted (C₁-C₂₀)hydrocarbyl. The thioimidate has the structure —C(SR¹)═N—.

If the variable X is —O—, then the imine —O—R¹ is a carboximidate. The carboximidate includes —O—R¹, wherein R¹ is substituted or unsubstituted (C₁-C₂₀)hydrocarbyl. The carboximidate has the structure —C(OR¹)═N—.

If the variable X is —NH—, then the imine —NH—R¹ is an aminoimidate. The aminoimidate includes —NH—R¹, wherein R¹ is substituted or unsubstituted (C₁-C₂₀)hydrocarbyl. The aminoimidate has the structure —C(NHR¹)═N—.

The amidine can have the structure —C(═NR²)—NH— and/or —C(—NHR²)═N—. The structures —C(═NR²)—NH— or —C(—NHR²)═N— can be tautomers of the amidine. The amidine can have the structure —C(—NR²₂)═N—. In the amidine, at each occurrence R² can independently chosen from H, —OH, substituted or unsubstituted (C₁-C₂₀)hydrocarbyl, substituted or unsubstituted (C₁-C₂₀)alkyl, substituted or unsubstituted (C₆-C₂₀)aryl, (C₆-C₂₀)heteroaryl, 3-halophenyl, 3-hydroxyphenyl, 3-(C₁-C₁₀)alkoxy-phenyl, 1,4-teterahydroxyoxazinyl, thiophenyl (C₁-C₁₀)alkyl, indolyl (C₁-C₁₀)alkyl, and pyridinyl (C₁-C₁₀)alkyl. The amidine can include ═NR², ═NH, —NH², —NHR², or —NR²₂ (e.g., the amidine can have the structure —C(═NH)—NH—, —C(—NH²)═N—, —C(═NR²)—NH—, —C(—NHR²)═N—, and/or —C (—NR²₂)═N—) wherein at each occurrence R² is independently chosen from —OH, substituted or unsubstituted (C₁-C₂₀)hydrocarbyl, substituted or unsubstituted (C₁-C₂₀)alkyl, substituted or unsubstituted (C₆-C₂₀)aryl, and (C₆-C₂₀)heteroaryl. The amidine can include —NH² or ═NH (e.g., the amidine can have the structure —C(═NH)—NH— and/or —C(—NH²)═N—). The amidine can include —NHR² or ═NR² (e.g., the amidine can have the structure —C(═NR²)—NH— and/or —C(—NHR²)═N—) or —NR²₂ (e.g., the amidine can have the structure C(—NR²₂)═N—), wherein R² is phenyl, benzyl, hydroxyl, or (C₁-C₂₀)alkyl. The amidine can include —N-R²₂ wherein the two R² groups together are a substituted or unsubstituted (C₂-C₂₀)hydrocarbyl that forms an aromatic or aliphatic ring that includes the nitrogen atom, wherein the hydrocarbyl group is optionally interrupted with 0, 1, 2, or 3 groups chosen from —O— and —S—. The product compound can be identical to the starting material compound other than the transformation of the imine —X—R¹ to the amidine.

In various aspects of the present invention, the formation of the amidine from the starting material compound and the nitrogen-containing reagent can be an intramolecular reaction: the nitrogen-containing reagent can be part of the starting material compound (i.e., they can be the same compound), and formation of the amidine can include an intramolecular ring-forming reaction. The nitrogen-containing reagent can include a primary amine or a secondary amine. The nitrogen-containing reagent can include a secondary amine that is part of an aromatic or aliphatic ring.

In various aspects of the present invention, the formation of the amidine from the starting material compound and the nitrogen-containing reagent can be an intermolecular reaction: the nitrogen-containing reagent can be a separate compound from the starting material compound. As compared to the amount of starting material compound present in the reaction milieu (e.g., the number of moles of imine —X—R¹ groups present), 1 to 20 equivalents of the nitrogen-containing compound can be present, or 1 to 10 equivalents, or less than or equal to 20equivalents and greater than or equal to 1 equivalent and less than, equal to, or greater than 1.5equivalents, 2, 2.5, 3, 4, 5, 6, 7, 8, 9, 10, 11, 12, 13, 14, 15, 16, 17, 18, or 19 equivalents. The nitrogen-containing reagent can include a primary amine; for example, the nitrogen-containing reagent can include aniline, benzylamine, hydroxylamine, or a combination thereof. The nitrogen-containing reagent can include a secondary amine; for example, the nitrogen-containing reagent can include piperidine, dimethylamine, or a combination thereof. The nitrogen-containing reagent can include an ammonium salt; for example, the nitrogen-containing reagent can include ammonium chloride, a bromide, iodide, acetate, nitrate, bicarbonate, nitrite, cyanide, amide, cyanate, a hydroxide, or a combination thereof. The ammonium salt can include ammonium chloride, ammonium acetate, or a combination thereof. The nitrogen-containing reagent can have the structure R²-NH₂ or R²₂-NH, wherein at each occurrence R² is independently chosen from —OH, substituted or unsubstituted (C₁-C₂₀)hydrocarbyl, substituted or unsubstituted (C₁-C₂₀)alkyl, substituted or unsubstituted (C₆-C₂₀)aryl, and (C₆-C₂₀)heteroaryl. The nitrogen-containing reagent can include ammonium acetate, ammonium chloride, aniline, benzylamine, piperidine, dimethylamine, hydroxylamine, or a combination thereof.

In various embodiments, the reaction of the starting material compound with the nitrogen-containing reagent is performed in the absence of added solvent. In other embodiments, the reaction of the starting material compound with the nitrogen-containing reagent is performed in an added solvent. The added solvent can be any suitable solvent. The solvent can include an organic solvent, such as a polar aprotic solvent. The solvent can include a halogenated solvent, chloroform, methylene chloride, tetrahydrofuran, dimethylformamide, 1,2-dimethyoxyethane, 1,3-dioxolane, dimethylsulfoxide, dimethyl acetamide, a fluoroalcohol, or a combination thereof. The solvent can include dimethylformamide, 2,2,2-trifluoroethanol, or a combination thereof. In various aspects, the added solvent includes dimethylformamide and 2,2,2-trifluoroethanol and includes a suitable volume ratio of the dimethylformamide to the 2,2,2-trifluorocthanol, such as 0.1:1 to 10:1, or 0.8:1 to 1.2:1, or less than or equal to 10:1 and greater than or equal to 0.1:1 and less than, equal to, or greater than 0.2:1, 0.3, 0.4, 0.5, 0.6, 0.7, 0.8, 0.9, 1, 1.1, 1.2, 1.3, 1.4, 1.5, 1.6, 1.8, 2, 2.5, 3, 3.4, 4, 4.5, 5, 6, 7, 8, or 9:1.

The added solvent can be substantially free of water. A reaction milieu including the starting material compound and the nitrogen-containing reagent (i.e., and including any added solvent) can be substantially free of water. For example, the reaction milieu can be 0 wt % to 2 wt % water, or 0 wt % to 0.1 wt % water, or less than or equal to 2 wt % water and greater than or equal to 0 wt % water and less than, equal to, or greater than 0.001 wt % water, 0.01, 0.1, 0.2, 0.4, 0.6, 0.8, 1, 1.2, 1.4, 1.6, or 1.8 wt % water.

In various aspects, the reaction milieu including the starting material compound and the nitrogen-containing reagent can be substantially free of added acids. In other aspects, the reaction milieu including the starting material compound and the nitrogen-containing reagent can further include an acid. During the conversion of the imine —X—R¹ to the amidine, the acid can protonate the imine —X—R¹. The acid can include any one or more suitable acids. The acid can include a carboxylic acid, a mineral acid, an organic acid, formic acid, a sulfonic acid, or a combination thereof. The acid can include acetic acid. Any suitable amount of acid can be used. For example, as compared to an amount of the starting material compound present in the reaction milieu (e.g., the number of moles of imine —X—R¹ groups present), 0.1 to 5 equivalents of the acid can be present, or 0.5 to 2 equivalents of the acid, or less than or equal to 5 equivalents and greater than or equal to 0.1 equivalents and less than, equal to, or greater than 0.2 equivalents, 0.4, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.5, 4, or 4.5 equivalents.

The reacting of the starting material compound and the nitrogen-containing compound can be performed at any suitable temperature. The reacting of the starting material compound and the nitrogen-containing reagent can be performed at room temperature, or 10° C. to 40° C., 15° C. to 30° C., or less than or equal to 40° C. and greater than or equal to 10° C. and less than, equal to, or greater than 12° C., 14, 16, 18, 20, 22, 24, 26, 28, 30, 32, 34, 36, or 38° C. The reacting of the starting material compound and the nitrogen can be performed at any suitable pressure, such as ambient pressure, or 20 kPa to 150 kPa, or 80 kPa to 120 kPa, or less than or equal to 150 kPa and greater than or equal to 20 kPa and less than, equal to, or greater than 30 kPa, 40, 50, 60, 70, 80, 90, 100, 110, 120, 130, or 140 kPa. The reacting of the starting material compound and the nitrogen-containing reagent can be performed for any suitable duration, such as 1 min to 72 h, or 1 h to 48 h, or less than or equal to 72 h and greater than or equal to 1 min and less than, equal to, or greater than 2 min, 4, 6, 8, 10, 15, 20, 30, 40, 50 min, 1 h, 2, 4, 6, 8, 10, 12, 14, 16, 18, 20, 22, 24, 26, 28, 30, 32, 34, 36, 38, 40, 42, 44, 46, 48, 50, 55, 60, 65, or 70 h.

The starting material compound including the imine —X—R¹ can be any suitable type of starting material compound. In various aspects, the starting material compound and the product compound are peptides, such as dipeptides, tripeptides, or polypeptides. The method can be performed as a solid phase synthesis or a solution phase synthesis.

The method can include performing the reacting of the starting material compound and the nitrogen-containing reagent while the starting material compound is bonded to a solid support. The method can further include bonding the starting material compound, or a precursor compound thereof, to the solid support (e.g., via one or more reactive groups thereof). The method can include performing one or more synthetic transformations of the precursor compound while the precursor compound is bonded to the solid support to form the starting material compound bonded to the solid support. The method can further include cleaving the product compound from the solid support.

The method can include performing the reacting of the starting material compound and the nitrogen-containing reagent while the nitrogen-containing reagent is bonded to a solid support (i.e., wherein R¹ is bonded to a solid support). The method can further include bonding the nitrogen-containing reagent, or a precursor compound thereof, to the solid support (e.g., via one or more reactive groups thereof). The method can include performing one or more synthetic transformations of the precursor compound while the precursor compound is bonded to the solid support to form the nitrogen-containing reagent bonded to the solid support. The method can further include cleaving the product compound from the solid support.

The method can further include isolating and/or increasing the purity of the product compound such as via any one or more purification and/or isolation methods, such as via chromatography.

The method can further include reacting a precursor compound including an X-amide (i.e., —C(═X)—NH—) to form the starting material compound including the imine —X—R¹ in place of the X-amide in the precursor compound. The starting material compound can be identical to the precursor compound other than the transformation of the X-amide to the imine-X-R¹. Reacting the precursor compound to form the starting material compound can include reacting the precursor compound with an alkyl iodide in the presence of a base.

The method can have any suitable yield of the product compound including the amidine from the starting material compound including the imine —X—R¹, such as a yield of 0.1% to 100%, or 40% to 99%, or less than or equal to 100% and greater than or equal to 0.1% and less than, equal to, or greater than 0.5, 1, 2, 3, 4, 5, 6, 8, 10, 15, 20, 25, 30, 35, 40, 45, 50, 55, 60, 65, 70, 75, 80, 85, 90, 91, 92, 93, 94, 95, 96, 97, 98, 99, 99.9, or 99.99%.

In the method, a reaction milieu including the starting material compound and the nitrogen-containing reagent can be substantially free of silver or silver-containing compounds. For example, silver or silver-containing compounds can be 0 wt % to 2 wt % of the reaction milieu including the starting material compound and the nitrogen-containing reagent, or 0 wt % to 0.1 wt %, or less than or equal to 2 wt % and greater than or equal to 0 wt % and less than, equal to, or greater than 0.001 wt %, 0.01, 0.1, 0.2, 0.4, 0.6, 0.8, 1, 1.2, 1.4, 1.6, or 1.8 wt %.

In various aspects, the product compound including the amidine can be a terminal product of the method, such that the product compound can be optionally isolated and purified. In other aspects, the product compound including the amidine can be an intermediate product that then reacts with other materials to form another product. In various aspects, the product compound can be isolated and then reacted with other materials to form another product. In various aspects, the product compound reacts in-situ with other materials to form another product compound.

For example, in various aspects, the method can further include reacting the amidine in the product compound intramolecularly with a carbonyl carbon in the product compound to form a cyclized product that includes a 4H-imidazolone and/or a derivative thereof. The method can include reacting the amidine with the carbonyl carbon to form the cyclized product in-situ in a same reaction container and/or same reaction mixture used to react the starting material compound with the nitrogen-containing reagent to form the product compound. The reaction conditions and/or reaction milieu used to form the cyclized product can be the same as reaction conditions and/or reaction milieu used to form the product compound.

In aspects of the present method including reacting the amidine in the product compound intramolecularly with the carbonyl carbon in the product compound to form the cyclized product, the product compound can include the structure:

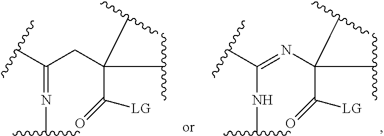

The cyclized product can include the structure:

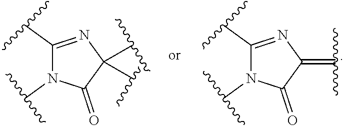 [SMILES: *C1=NC(*)(*)C(=O)N1* ; *C1=NC(=*)C(=O)N1*]

The variable LG is a leaving group.

In aspects of the present method including reacting the amidine in the product compound intramolecularly with the carbonyl carbon in the product compound to form the cyclized product, the product compound can be R²—N^{A}═CR³—NH—CR⁴R⁵—C^{A}(O)—LG or R²—N^{A}H—CR³═N—CR⁴R⁵—C^{A}(O)—LG. The method can include reacting the amidine intramolecularly with the carbonyl carbon includes reacting the nitrogen atom N^{A} with the carbonyl carbon C^{A}.

The variable R³ can be chosen from —H, —OH, —OR⁶, substituted or unsubstituted (C₁-C₂₀)hydrocarbyl, substituted or unsubstituted (C₁-C₂₀)alkyl, substituted or unsubstituted (C₆-C₂₀)aryl, and substituted or unsubstituted (C₆-C₂₀)heteroaryl. The variable R³ can be chosen from phenyl, benzyl, hydroxyl, (C₁-C₂₀)alkyl, and derivatives and substituted variations thereof.

At each occurrence, the variable R⁶ can be independently chosen from substituted or unsubstituted (C₁-C₂₀)hydrocarbyl, substituted or unsubstituted (C₁-C₂₀)alkyl, substituted or unsubstituted (C₆-C₂₀)aryl, and substituted or unsubstituted (C₆-C₂₀)heteroaryl. The variable R⁶ can be independently chosen from (C₁-C₂₀)hydrocarbyl, (C₁-C₂₀)alkyl, and (C₆-C₂₀)aryl.

The variables R⁴ and R⁵ can be independently chosen from —H, —R⁶, —OH, —NH₂, —NHR⁶, —OR⁶, and —SR⁶. The variables R⁴ and R⁵ can be independently chosen from —H, substituted or unsubstituted (C₁-C₂₀)alkyl, and substituted or unsubstituted (C₆-C₂₀)aryl. The variables R⁴ and R⁵ can be independently chosen from —H and (C₁-C₅)alkyl. The variables R⁴and R⁵ can be —H. The variables R⁴ and R⁵ can be methyl.

The variable LG can be any suitable leaving group. The leaving group can be chosen from halide, —OR⁶, and an O-ester. The leaving group can be a suitable leaving group associated with peptide chemistry and/or a group that promotes nucleophilic substitution. The leaving group can be —OCH₃.

The cyclized product can have the structure:

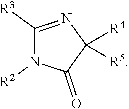

In various aspects, the reaction of the amidine forms an intermediate compound that goes on to react to form a 4H-imidazolone and/or a derivative thereof. For example, the reaction of the amidine can form an intermediate having the structure:

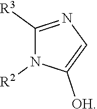

The method can further includes reacting the intermediate with an electrophile to form the cyclized product having the structure:

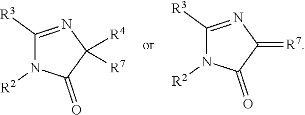

The variable R⁷ is a product of reaction of the electrophile with the intermediate. In various aspects, the reaction of the intermediate with the electrophile is performed in-situ in a same reaction container and/or reaction mixture used to react the amidine intramolecularly with the carbonyl carbon.

The method can include performing the reacting of the amidine intramolecularly with the carbonyl carbon under acidic conditions. For example, the acidic conditions can include a reaction milieu that includes an organic acid, a mineral acid, or a combination thereof. In various aspects, the reaction milieu can include acetic acid or pyridinium p-toluenesulfonate (PPTS).

In various aspects, the formed 4H-imidazolone cyclized product includes a cis-amide. The trans-amide is typically thermodynamically more stable than the cis-amide; as such, various aspects can give access to the less thermodynamically stable and more difficult to access cis-amide product. For example, in various aspects, 100% of the cyclized product can be the cis-amide, or 50% to 100%, or less than or equal to 100% and greater than or equal to 50% and less than, equal to, or greater than 55%, 60, 65, 70, 75, 80, 85, 90, or 95%.

The reacting of the product compound to form the cyclized product can be performed neat or using a concentration of the product compound in a reaction solvent of 0.01 M to 10 M, 0.1 M to 5M, or 0.5 M to 0.15 M, or less than or equal to 10 M and greater than or equal to 0.01 M and less than, equal to, or greater than 0.05, 0.1, 0.15, 0.2, 0.3, 0.4, 0.5, 0.6, 0.8, 1, 1.5, 2, 2.5, 3, 4, 5, 6, or 8 M.

The reacting of the product compound to form the cyclized product can be performed at a reaction temperature of 0° C. to 100° C., or 20° C. to 60° C., or less than or equal to 100° C. and greater than or equal to 0° C. and less than, equal to, or greater than 5° C., 10, 15, 20, 25, 30, 35, 40, 45, 50, 55, 60, 65, 70, 75, 80, 85, 90, or 95° C. The method can include maintaining the reaction temperature for any suitable duration, such as a duration of 30 min to 48 h, or 10 h to 30 h, or less than or equal to 48 h and greater than or equal to 30 min and less than, equal to, or greater than 1 h, 2, 3, 4, 5, 6, 8, 10, 12, 14, 16, 18, 20, 22, 24, 26, 28, 30, 32, 34, 36, 38, 40, 42, 44, or 46 h.

The method can include forming the cyclized product from the product compound at a yield of 20% to 100%, or 60% to 100%, or less than or equal to 100% and greater than or equal to 20% and less than, equal to, or greater than 25%, 30, 35, 40, 45, 50, 55, 60, 62, 64, 66, 68, 70, 72, 74, 76, 78, 80, 82, 84, 86, 88, 90, 91, 92, 93, 94, 95, 96, 97, 98, or 99%. The method can include forming the cyclized product from the product compound at a conversion of the product compound of 50% to 100%, or 80% to 100%, or less than or equal to 100% and greater than or equal to 50% and less than, equal to, or greater than 55%, 60, 62, 64, 66, 68, 70, 72, 74, 76, 78, 80, 82, 84, 86, 88, 90, 91, 92, 93, 94, 95, 96, 97, 98, or 99%.

The method of forming the cyclized product from the product compound can include performing the reacting of the product compound to form the cyclized product while the product compound is bonded to a solid support. The method can further include cleaving the cyclized product from the solid support.

### Method of Intramolecular Reaction of Amidines to Form 4H-Imidazolones.

Various aspects of the present invention provide a method of forming a cyclized product from a compound that includes an amidine. The amidine can be generated in any suitable way, such as via the presently disclosed method for forming the product compound (e.g., the compound that includes the amidine can be the product compound), or such as via a different method. The method can include intramolecularly reacting a compound that includes the structure:

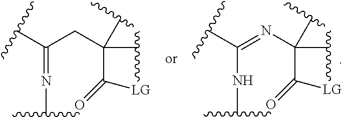

The intramolecular reaction forms a cyclized product including the structure:

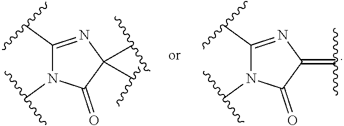 [SMILES: *C1=NC(*)(*)C(=O)N1* ; *C1=NC(=*)C(=O)N1*]

The variable LG is a leaving group.

### EXAMPLES

Various aspects of the present invention can be better understood by reference to the following Examples which are offered by way of illustration. The present invention is not limited to the Examples given herein.

Materials. Unless otherwise specified, all commercial products and reagents were used as purchased, without further purification. Solvents were reagent grade. Solvents including, THF, DCM, and DMF were dried via a Glass Contours Inc. solvent purification system. Analytical thin-layer chromatography (TLC) and flash chromatography of all reactions were performed on silica gel (40 μm) purchased from Grace Davison.

NMR Spectroscopy. ¹H and ¹³C NMR spectra for all compounds were acquired in deuterated solvents (as indicated) on a Bruker Spectrometer at the field strengths reported in the text. The chemical shift data are reported in units of δ (ppm) relative to the residual solvent.

Liquid Chromatography. LC-HRMS data was collected Agilent QTOF 6540MSMS HPLC system with an Agilent XDB C18 column 1.8 μm. HPLC solvent contained water with 0.1% Formic Acid and acetonitrile with 0.1% Formic acid.

Peptide isolation and analysis were accomplished using an Xbridge Prep OBD C18 column (5 μm) on a Water HPLC preparative LC system equipped with detection using a 2998 Photoiodiode Array Detector. Purification occurred with water and acetonitrile containing 0.1% TFA (v/v) as eluents.

Mass Spectrometry. HRMS data of peptides were collected on an Agilent QTOF 6540 MSMS with ESI ionization and TOF detection. Chemical Formulas found from HRMS data were obtained via Agilent MassHunter software equipped with the HRMS QTOF.

Synthesis of thioacylating reagent Fmoc—Xₐₐ^{(S)}—Nbt was carried out according to previous procedures with matching spectra.

### Example 1. Conversion of Thioimidate Into Amidine

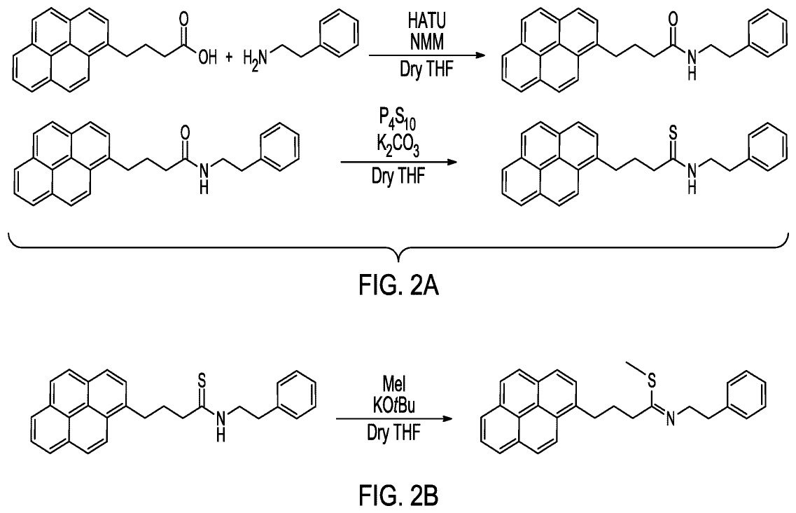

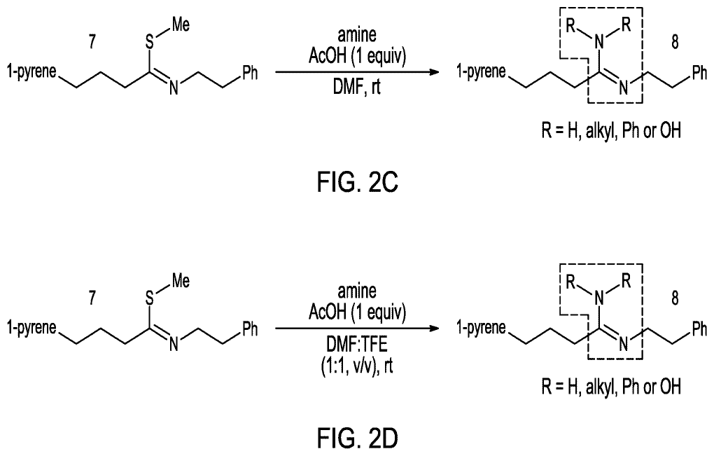

To determine the feasibility of installing an amidine via the thioimidate in linear peptides, we began with compound 7 as a simple test substrate that was free of any other competing functional groups (Table 1). FIG. 2A illustrates the synthesis of N-phenylethyl-4-(pyren-4-yl) butanethioamide. FIG. 2B illustrates the synthesis of N-phenylethyl-4-(pyren-4-yl) butanethioimidate (7). The thioimidate was found highly amenable to conversion into amidine (8) with weak acid in DMF, conditions that are compatible with SPPS procedures, as shown in FIG. 2C. It should be noted that under basic conditions related to SPPS, the thioimidate is nonreactive towards amine nucleophiles and no amidine formation has been observed. Presumably, the acetic acid creates a mildly basic buffer with the amine, where protonation of the thioimidate (pkₐ=7.5-8) thereby increases its electrophilicity towards nucleophilic substitution with the amine. Notably, less nucleophilic amines such as aniline (entry 5) were less efficient in forming amidine relative to primary amines (entries 1-4). Likewise, more sterically hindered secondary amines (entries 6 and 7) provided only modest to good yields of amidine, even after extended reaction times. Finally, primary amidines were accessed from ammonium salts in only modest yields (entries 8 and 9). The addition of acetic acid was not found to have any effect, likely due to the presence of the internal acid of the ammonium cation. While ammonia is a weaker nucleophile than primary amines, this result was at first surprising because ammonia appeared to be even less efficient than aniline (entry 8 and 9 versus 5).

TABLE 1
Conversion of thioimidate into amidine

| entry | amine | equivalents | time (h) | yield (%) |
|---|---|---|---|---|
| 1 | BnNH₂ | 10 | 2 | 99 |
| 2 | BnNH₂ | 5 | 2 | 98 |
| 3 | BnNH₂ | 1 | 2 | 92 |
| 4 | H₂N—OH | 10 | 0.5 | 95 |
| 5 | PhNH₂ | 10 | 6 | 65 |
| 6 | Me₂NH | 10 | 18 | 85 |
| 7 | piperidine | 10 | 24 | 54 |
| 8 | NH₄Cl ^{a} | 5 | 3 | 56 |
| 9 | NH₄OAc ^{a} | 2 | 3 | 34 |

Pyrene was used as internal standard.
^{a} No AcOH was added.
% Yield values are the average of two experiments.

To synthesize N-phenylethyl-4-(pyren-4-yl) butanethioamide, as illustrated in FIG. 2A 1-pyrenebutyric acid (1.00 g, 3.5 mmol) was dissolved in dry DMF in a round bottom flask and was cooled to 0° C. in an ice bath. To the chilled flask, HATU (1 eq., 1.33 g, 3.5 mmol) and NMM (3 eq., 1.1 mL, 10.5 mmol) were added, and the mixture was allowed to stir on ice for 5 min. 2-Phenylethan-1-amine (1.25 eq., 5.5 mL, 4.3 mmol) was added, and the reaction was allowed to stir for 2 h in an ice bath. The reaction was diluted with 75 mL of EtOAc and the organic portion was washed with 1 M HCl, NaHCO₃, and brine (3×30 mL, each). The organic layer was dried over sodium sulfate, filtered, and concentrated under reduced pressure to yield 1.26 g (80%) of an off-white solid that was used without any further purification in the next step.

P₄S₁₀ (2.85 g, 6.2 mmol) and K₂CO₃ (1.33 g, 9.3 mmol) were placed in a round bottom with 60 mL of dry THF and stirred at rt for 20 min. To the same flask, the amide (from above, 1.26 g, 3.2 mmol) was added, and the mixture was refluxed and monitored via TLC. After 4 h the solid precipitate was filtered off, and the filtrate was concentrated under reduced pressure to yield crude product. The product was purified via silica gel chromatography (3:1, hexanes/EtOAc w/0.1% DIEA (v/v)) to yield 1.25 g (95%) of a yellow oily solid. ¹H NMR (400 MHz, CDCl3) δ 8.39 (d, J=9.2 Hz, 1H), 8.31 (d, J=7.6 Hz, 2H), 8.23 (dd, J=8.5, 6.1 Hz, 2H), 8.18-8.12 (m, 3H), 7.93 (d, J=7.8 Hz, 1H), 7.44-7.39 (m, 2H), 7.38-7.33 (m, 1H), 7.32-7.28 (m, 2H), 4.06-3.99 (m, 2H), 3.46 (t, J=7.6 Hz, 2H), 3.03 (t, J=7.1 Hz, 2H), 2.78 (t, J=7.4 Hz, 2H), 2.51-2.41 (m, 2H). ¹³C NMR (101 MHz, CDCl3) δ 205.06, 138.15, 135.65, 131.44, 130.92, 129.99, 128.80, 128.76, 128.69, 127.53, 127.45, 127.32, 126.81, 125.97, 125.10, 125.02, 124.99, 124.87, 123.37, 46.73, 46.36, 33.70, 32.28, 30.85. HRMS: Found [M+H] 408.1771 m/z, calculated for C₂₈H₂₆NS⁺ 408.1781 m/z.

To synthesize N-phenylethyl-4-(pyren-4-yl) butanethioimidate (7), as illustrated in FIG. 2B thioamide (600 mg, 2.23 mmol) was dissolved in 3 mL of dry DMF in a round bottom flask. The flask was cooled to 0° C. in an ice bath before potassium tert-butoxide (300 mg, 2.67 mmol) was added. The mixture was stirred for ca. 5 min. and methyl iodide (210 μL, 3.34 mmol) was added dropwise. The resulting mixture was allowed to continue stirring on ice (with gradual warming to rt) over 2 h. The reaction was quenched with saturated ammonium chloride solution and extracted with DCM (3×30 mL). The organic layers were combined and dried over sodium sulfate, filtered, and concentrated in vacuo to yield 575 mg (92%) of an orange oily solid. ¹H NMR (600 MHz, CDCl₃) inseparable rotamers: δ 8.40 (d, J=9.2 Hz, 1H), 8.25 (d, J=9.2 Hz, 1H), 8.24-8.20 (m, 4H), 8.18-8.12 (m, 4H), 8.09-8.03 (m, 6H), 7.91 (d, J=7.8 Hz, 1H), 7.85 (d, J=7.8 Hz, 1H), 7.42-7.36 (m, 4H), 7.31-7.21 (m, 4H), 7.18-7.15 (m, 2H), 3.75-3.71 (m, 2H), 3.63 (t, J=7.4 Hz, 2H), 3.48-3.44 (m, 2H), 3.36 (t, J=7.8 Hz, 2H), 3.21-3.17 (m, 2H), 2.96 (t, J=7.4 Hz, 2H), 2.68 (t, J=7.1 Hz, 2H), 2.50-2.46 (m, 2H), 2.42 (s, 2H), 2.28 (s, 5H), 2.15-2.09 (m, 2H). ¹³C NMR (150 MHz, CDCl₃) inseparable rotamers: δ 166.99, 163.99, 140.55, 140.38, 136.42, 135.56, 131.45, 130.96, 130.91, 129.98, 129.90, 128.95, 128.83, 128.65, 128.37, 128.24, 127.54, 127.42, 127.41, 127.25, 127.16, 126.74, 126.66, 126.08, 126.01, 125.88, 125.84, 125.10, 125.04, 125.02, 124.98, 124.89, 124.82, 124.75, 123.62, 123.15, 68.01, 54.19, 53.21, 37.89, 37.14, 36.93, 33.76, 32.99, 32.67, 29.01, 29.00, 25.65, 14.01, 12.65. HRMS: Found [M+H] 422.1947 m/z, calculated for CHNS⁺ 422.1937 m/z.

For each entry in Table 1, compound 7 (25 mg, 0.06 mmol) was dissolved in dry DMF along with 1 eq. of pyrene, as the internal standard. Acetic acid and amine reaction partner were added to the reaction as indicated by entries 1-9 in Table 1. Each reaction was stirred at rt and monitored via LS-HRS to identify the amidine product. Percent yield was calculated based on the absorbance of starting material and product relative to the absorbance of the pyrene internal standard at 330 nm.

### Example 2. Conversion of Thioimidate Into Amidine in the Presence of Fluoroalcohol

We hypothesized that the lower yields in Example 1 were due primarily to low solubility of the ammonium salts in DMF. To test this hypothesis, trifluoroethanol (TFE) was included in the solvent mixture (Table 2), as shown in FIG. 2D. The modified solvent conditions afforded increase solubility of ammonium salts, leading to homogenous solutions with significantly improved yields (entries 1 and 2). Moreover, the addition of TFE was found to improve the yields and reaction times of alkyl amines (entry 3), including amines with lower nucleophilicity due to both electronic (entry 4) or steric (entry 5) effects relative to neat DMF (Table 1). Other fluorinated alcohols like the more expensive hexafluoroisopropanol (HFIP) were found to have a similar results as TFE. The beneficial effects of TFE obviously extend beyond simple solubility, wherein the strong hydrogen-bond donor ability of fluoroalcohols may help to meditate the shuttling of protons during the reaction.

TABLE 2
Formation of amidine with the addition of fluoroalcohol.

| entry | amine | equivalents | time (h) | yield (%) |
|---|---|---|---|---|
| 1 | NH₄Cl^{a} | 5 | 3 | 98 |
| 2 | NH₄OAc^{a} | 2 | 3 | 99 |
| 3 | BnNH₂ | 10 | 1 | 98 |
| 4 | PhNH₂ | 10 | 3 | 99 |
| 5 | piperidine | 10 | 3 | 75 |

Pyrene was used as internal standard.
^{a} No AcOH was added.
% Yield values are the average of two experiments.

For each entry in Table 2, compound 7 (25 mg, 0.06 mmol) was dissolved in dry DMF along with 1 eq. of pyrene, as the internal standard. Acetic acid and amine reaction partner were added to the reaction as indicated by entries 1-5 in Table 2. Each reaction was stirred at rt and monitored via LS-HRS to identify the amidine product. Percent yield was calculated based on the absorbance of starting material and product relative to the absorbance of the pyrene internal standard at 330 nm.

### Example 3. Conversion of Thioimidate Into Amidine Along a Peptide Backbone and on Solid Support

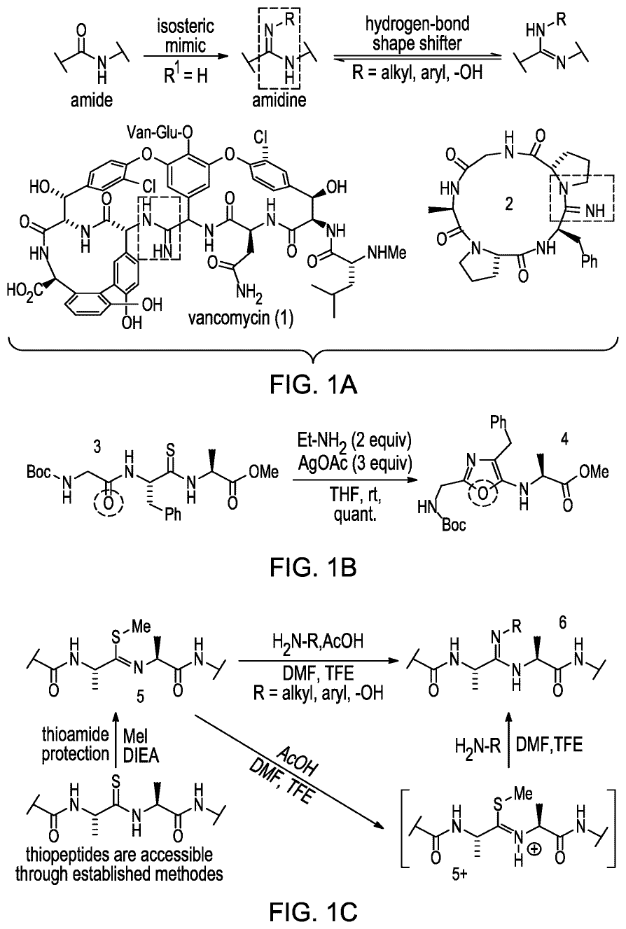

We next sought to explore the feasibility of this reaction along a peptide backbone and on solid support. We used the trimer Phe^{SMe}-Ala-Ala as a test sequence, where the thioimidate was placed at the linkage between the Phe and Ala residues (Table 3). Notably, this test trimer positioned an oxoamide to either side of the thioimidate linkage. Thus, the test trimer allowed us to explore the possibility that the oxoamide to either the N-terminal or C-terminal side of the thioimidate might react and cyclize onto the thioimidate to form oxazole or other products (akin to the side reaction observed with thiopeptides activated with Ag(I), FIG. 1B).

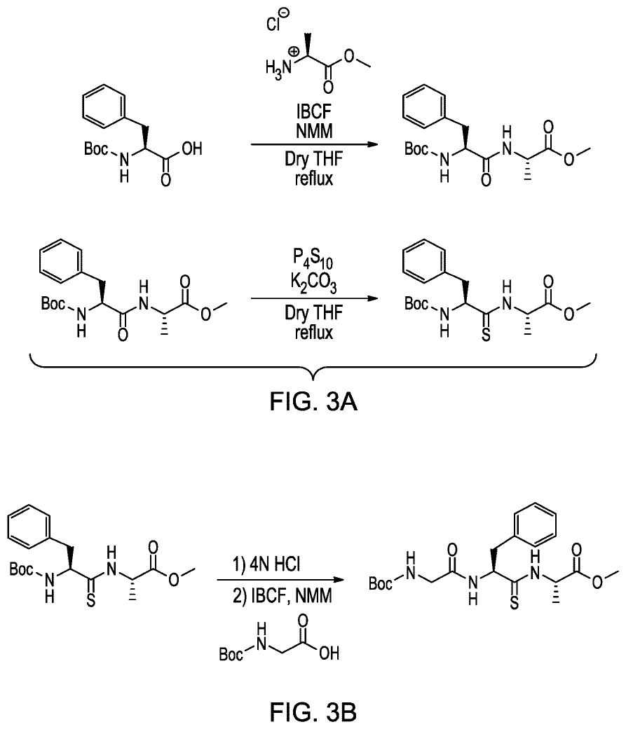

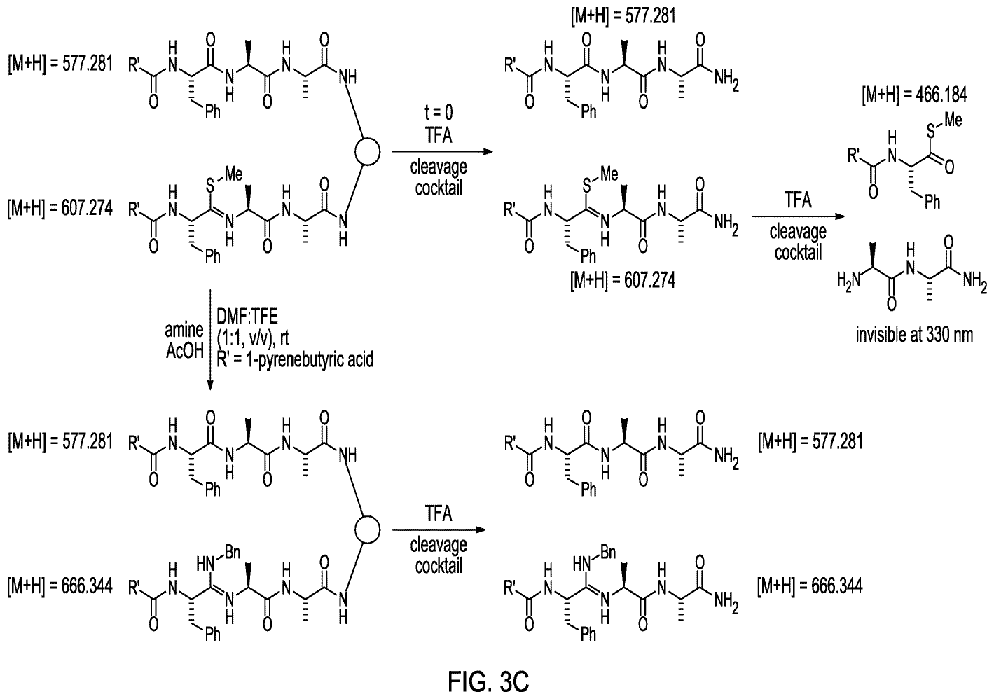

To synthesize Boc-Phe(S)-Ala-OMe dipeptide, as illustrated in FIG. 3A, based on a previously reported procedure, Boc-L-phenylalanine (1.33g, 5.00 mmol) was dissolved in dry THF and cooled to 0° C. in an ice bath. Isobutyl chloroformate (0.650 mL, 5.5 mmol, 1.1 eq.) was added dropwise, followed by the dropwise addition of NMM (2.2 mL, 20 mmol, 4 eq.) to produce a white precipitate. The mixture stirred for 10 min. after which solid L-Ala-OMe hydrochloride salt (0.517 g, 1.1 eq.) was added in one portion. The mixture stirred overnight while gradually warming to r.t. and the white precipitate was filtered. The filtrate was diluted with EtOAc and washed with 0.1M HCl, sodium bicarbonate, and brine (50 mL, each). The organic layer was dried over sodium sulfate, filtered and the filtrate was concentrated in vacuo to yield 1.4 g (84%) of Boc-Phe-Ala-OMe dimer, used without any further purification in the next step. Spectral data matched previous reports. P₄S₁₀ (2.79 g, 6.3 mmol) and K₂CO₃ (1.73 g, 12.5 mmol) were placed in a round bottom and dissolved in 100 mL of dry THF, stirred at rt for 20 min. Boc-Phe-Ala-OMe (1.467 g, 4.18 mmol) was added to the flask, and the mixture was heated under reflux for 4 h indicated by the consumption of starting material by TLC. The reaction was then cooled, filtered, and the filtrate was concentrated in vacuo. The product was purified via silica chromatography (1:2, EtOAc/Hexanes) to yield 1.5 g (56%) of a pale-yellow solid. Spectral data matched previous reports.

To synthesize Boc-Gly-Phe(S)-Ala-OMe tripeptide (3), as shown in FIG. 3B, Boc-Phe(S)-Ala-OMe (375 mg, 1 mmol) was dissolved in minimal dioxane in and added to a flame dried flask (in an ice bath) containing 10 mL of 4N HCl. The resulting mixture was allowed to stir while gradually warming to r.t. for ca. 45 min. which after is concentrated under reduce pressure. The crude “sludge” was redissolved in THF and neutralized with NMM to produce a white precipitate. The precipitate was filtered, and the filtrate was concentrated under reduced pressure. This process was repeated once more to yield crude free amine. The free amine was redissolved in dry THF and Boc-Gly (179 mg, 1.00 mmol, 1 eq.), NMM (.45 mL, 4 mmol) and IBCF (.135 mL, 1.00 mmol) was added to a flask and cooled to 0° C. in an ice bath. The mixture was stirred while gradually warming overnight, and then the white precipitate was filtered. The filtrate was diluted with EtOAc and washed with .1 M HCl, sodium bicarbonate, and brine (all, 3×25 mL). The organic layer was dried over sodium sulfate, filtered, and the filtrate was concentrated in vacuo to yield crude tripeptide. The crude product was purified via column chromatography (1:2 Hexanes/EtOAc) to yield 348.5 mg (80%, over 2 steps) pale-yellow solid. ¹H NMR (400 MHz, CDCl₃) δ 8.40 (d, J=7.0 Hz, 1H). 7.32-7.12 (m, 5H), 5.21 (t. J=5.7 Hz, 1H). 5.05 (q, J=7.3 Hz, 1H), 4.93 (p, J=7.1 Hz, 1H), 3.80 (t. J=6.1 Hz, 2H). 3.70 (s, 3H) 3.19 (dd, J=13.4, 6.5 Hz. 1H), 3.08 (dd, J=13.5, 7.3 Hz. 1H), 1.47 (s, 9H). 1.41 (d. J=7.7 Hz. 3H). ¹³C NMR (100 MHz, CDCl₃) δ 202.30, 171.86, 171.29, 169.15, 136.31, 129.42, 128.65, 127.15. 80.52, 77.48, 77.16, 76.84, 60.52, 60.21, 53.54, 52.69, 44.54, 42.10, 28.43. 21.17, 16.97, 14.32.

General Solid Phase Peptide Synthesis Procedures. A Chemglass Peptide Synthesis Vessel was supplied with Tentagel S RAM resin (.23 mmol/g loading) that was swelled in dry DMF for 15 min. with nitrogen agitation. After swelling, the Fmoc protecting group (and all others going forward) was deprotected with 20% piperidine in dry DMF twice (2 min., then 8 min.). After deprotection of Fmoc, the resin was washed with dry DMF (5×1 min.). Coupling of all oxo-amide residues was achieved by dissolving Fmoc-Xaa-OH (5 eq.) (and all amide residues going forward), HATU (4.9 eq.), and NMM (10 eq.) in dry DMF to a concentration of 100 mM, with respect to the amino acid. This solution was added to the resin and agitated with bubbling N2 for 30 min. After each coupling, the resin was washed with dry DMF (3×1 min.).

Thioamide Coupling. The thioamide residue is introduced by dissolving 2 eq. of thioacylating reagent (Fmoc-X_{a.a}^{(S)}-Nbt) in dry DCM with DIEA (2 eq.). This solution was agitated with the resin using nitrogen for 30 min. and repeated a second time. Following the completion of the second coupling, the resin was washed with dry DCM (3x 1 min.). The resin was capped with Ac₂O (5 eq.) and NMM (5 eq.) to cap any unreacted peptide after the addition of the thioamide. The Fmoc group is not removed at this stage.

Thioamide Protection. The resin was subjected to a mixture of 0.5 M methyl iodide (Mel) and 0.05 M DIEA in dry DMF and agitated for 6 hr. on a rotary mixer. Then solvent was drained and a fresh mixture of Mel and DIEA was added. The resin was agitated for an additional 6 hr. on the rotary mixer. Test cleavage was performed to confirm methylation was complete, and upon the presence of thioamide detected via MS, additional methylation was required. Upon full methylation of the thioamide, the Fmoc was deprotected as described above, and subsequent peptide elongation was performed. The final residue was acetyl-capped using Ac₂O (10 eq.) and NMM (5 eq.) in dry DMF.

Amidine Installation. After completion of the peptide, the resin was subjected to a mixture of 5 M Amine, 1 M Acetic Acid in a solvent mixture of DMF: TFE (1:1 v/v). The resin was agitated on a rotary mixer for 24 hours, with fresh reagent being introduced ca. every 8 hours. Test cleavage was performed to confirm that the thioimidate fully reacted using MS detection. The resin was further introduced to the reaction mixture if there was still the presence of thioimidate.

Isolation and purification. The peptide was cleaved from resin with the cleavage cocktail, 95/2.5/2.5% (% v/v) TFA, water, and TIPS, respectively. The resin was subjected to the cocktail twice for 30 minutes. The cocktail was removed into a falcon tube (Tube A), where it was evaporated with a gentle stream of air until a thin film was reached. To the falcon tube, cold ether was added, and Tube A was centrifuged at 10,000 rpm for 5 min. This was repeated with new cold ether added to Tube A to yield crude peptide as a small pellet. The peptide was redissolved in a solution of MeCN (60%), H₂O (40%), and 0.1% TFA. The peptide was then purified via HPLC using solvent gradients. Percent yield was calculated based on the absorbance of starting material and product relative to the absorbance of the all-oxoamide (577.281) signal an internal standard at 330 nm.

TABLE 3
Amidine formation from thioimidate during
solid-phase peptide synthesis.

| entry | amine | [Amine] | [Acetic Acid] | yield (%) ^{b} |
|---|---|---|---|---|
| 1 | NH₄OAc^{a} | 5 | — | 93 |
| 2 | BnNH₂ | 5 | 1 | 48 |
| 3 | BnNH₂ | 2 | .5 | 22 |
| 4 | PhNH₂ | 5 | 1 | 44 |
| 5 | piperidine | 5 | 1 | trace |

^{a} no AcOH was added.
^{b} % Yield of amidine determined relative to a R′-FAA-NH₂ internal resin standard using LC-HRMS and monitoring at 330 nm.
Identity of the amidine confirmed via HRMS.

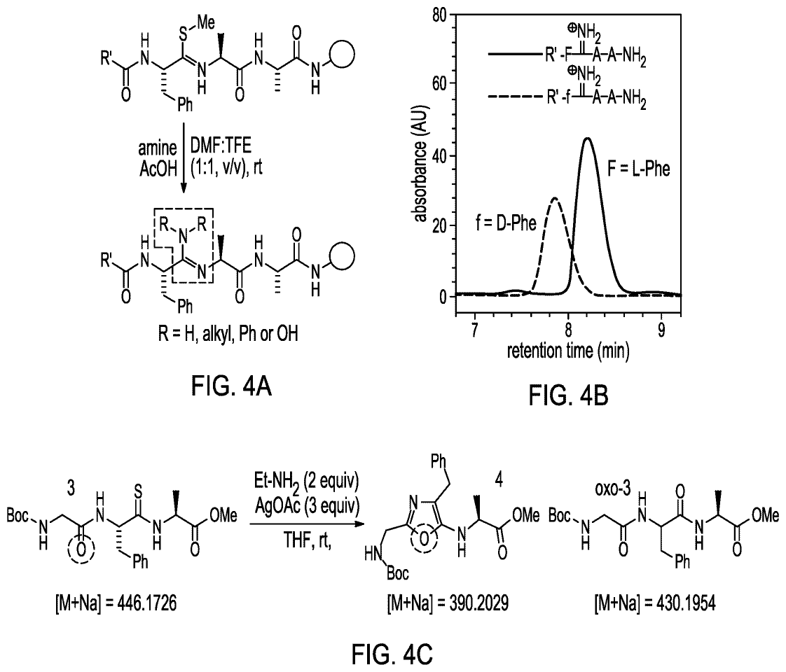

Using conditions that were derived from the results in Table 2, amidines were found to incorporate at the thioimidate site in serviceable yields after 24 h at room temperature (Table 3, entries 1-5, FIG. 4A). We found a 5:1 ratio of molarity of the alkyl amine to AcOH to be a generally applicable condition for amidine formation (entries 2 and 5). Higher concentrations of AcOH relative to benzyl amine (e.g. 5:5 or 5:2.5 M for BnNH₂:AcOH) led to a salting out of benzylammonium acetate from solution. Lower concentrations of amine and AcOH produced lower yields (entries 3 and 4). Higher concentrations of acid may provide optimal yields for other amine nucleophiles, which certainly appears to be the case for the 1:1 molar equivalents of ammonia and acetic acid in NH₄OAc (entry 1), but the general ratio of 5:1 M amine:AcOH was used to prepare the peptides described below.

To evaluate the possibility for epimerization of the α-C stereochemistry of the Phe residue during the reaction, we compared the results in entry 1 and entry 3 to analogous trimers prepared with authentic D-Phe (FIG. 4B). These results did not indicate epimerization to be a major concern as the two epimers were distinctly separated by chromatography. The results reported in Tables 1 and 2 indicated that more sterically hindered secondary amines were more sluggish nucleophiles in the reaction. This behavior appears to be exacerbated on-resin as piperidine failed to give productive yields of corresponding amidine (entry 6). Importantly, in all cases, we did not observe products associated with oxazole-cyclized side product (FIG. 1B). In fact, in all cases, no other unexpected species were observed besides the expected products and any unreacted starting material, indicating a clean conversion process. Finally, the ability to introduce amidines directly on-resin, while side-chain functional groups remain in protected form, increases the sequence applicability of the synthetic approach.

FIG. 4C illustrates rapid oxazole formation during amidine formation from thioamides using silver catalyst. Compound 3 (100 μmol), DIEA (17.4 μL, 2 Eq, 200 μmol), silver acetate (17.5 mg, 105 μmol), ethyl amine (2 eq.) and diphenyl ether (as internal standard, 2 eq.) were dissolved in 0.5 mL THF. The reaction was analyzed via HPLC-MS as shown below. Compound oxo-3 arises due to facile hydrolysis of oxazole 4, so any observation of oxo-3 implies formation of 4.

### Example 4. Preparation of Biologically Relevant Peptides

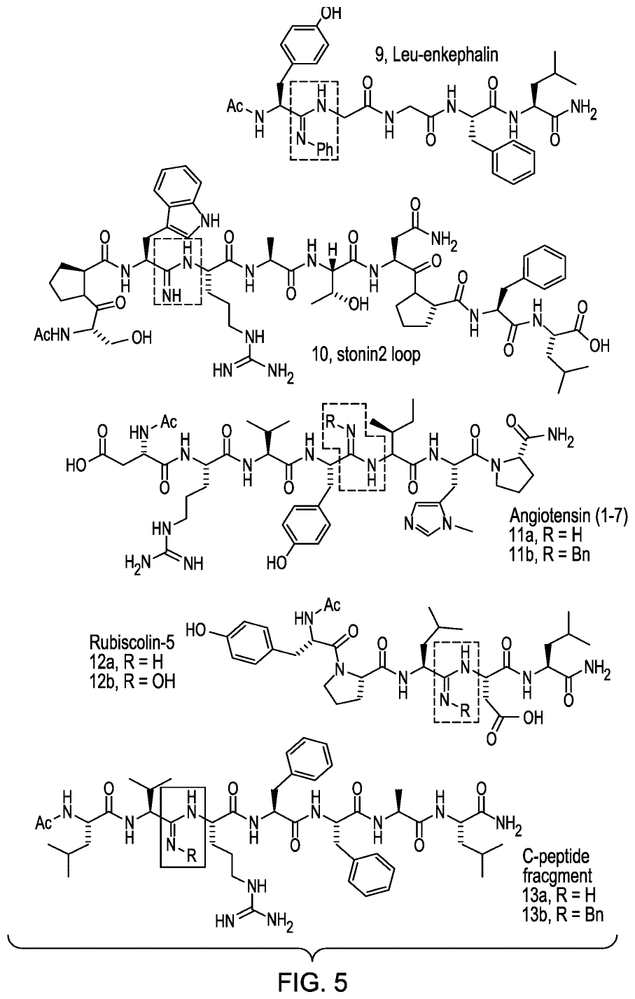

We next prepared a range of biologically relevant peptides to showcase the generality of using the thioimidate as a site for selective insertion of amidines (FIG. 5). Peptides were synthesized by insertion of the thioimidate following established procedures, followed by subsequent elongation to complete the sequence. Conversion to the amidine took place prior to final cleavage from the resin following the procedure described in Example 3.

The Leu-enkephalin pentapeptide (9) is a selective agonist for δ-opioid receptors, with potential to treat chronic pain and inflammation without eliciting respiratory depression and addiction. Despite these promising attributes, Leu-enkephalin has a poor pharmacokinetic profile due to rapid proteolysis of the Tyr-Gly linkage by aminopeptidase N in human plasma. Installation of an aniline amidine at this key metabolic site was readily achieved using our approach.

The Eps15 protein is involved in a multiprotein complex with stonin2 to recruit machinery necessary for endocytosis. It was discovered that a truncated loop of stonin2, peptide (10), makes several key contacts with Eps15. We were successful in insertion of an amidine between two amino acids with (Trp-Arg) possessing larger side chains, one of them charged.

The angiotensin (1-7) peptide binds and activates G-protein coupled Mas receptors with applications in pain management. New analogues of angiotensin (1-7) with unexplored biodistribution and pKa profiles may be possible through modification of the peptide backbone (11a and 11b). 11a, R═H: HRMS: Found 853.5315 m/z, 477.7675 m/z, and 318.5149 m/z, calculated for C₄₄H₆₈N₁₄O₁₀⁺ 953.5316 m/z, C₄₄H₆₈N₁₄O₁₀⁺² 477.2694 m/z, and C₄₄H₆₈N₁₄O₁₀⁺³ 318.5154 m/z. 11b, R=Bn: HRMS: Found 1043.5809 m/z, 522.2937, and 348.513 m/z calculated for C₅₁H₇₄N₁₄O₁₀⁺ 1043.5785, C₅₁H₇₄N₁₄O₁₀⁺² 522.2929, and C₅₁H₇₄N₁₄O₁₀⁺³ 348.5310 m/z.

Rubiscolin-5 is a naturally occurring peptide agonist with high selectivity against δ-opioid receptors over other signaling receptors. New classes of rubiscolin-5 compounds were accessed through modification of the polyamide backbone (12a and 12b). 12a, R═H: HRMS: Found 660.3719 m/z, calculated for C₃₂H₄₉N₇O₈⁺ 660.3715 m/z. 12b, R═NH—OH: HRMS: Found 676.3674 m/z, calculated for C₃₂H₄₉N₇O₉⁺ 676.3665 m/z.

Finally, amidine installation was also possible at bulky, β-branched amino acids such as the valine residue of a truncated sequence of insulin-related C-peptide (13a and 13b). 13 a, R═H: HRMS: Found 792.4864 m/z and 396.7462, calculated for C₄₀H₆₁N₁₁O₆⁺ 792.4870 m/z and C₄₀H₆₁N₁₁O₆⁺² 396.7476 m/z. 13b, R═Bn: HRMS: Found 882.5333 and 441.7715 m/z, calculated for C₄₇H₆₇N₁₁O₆⁺ 882.5349 m/z and C₄₇H₆₇N₁₁O₆⁺² 441.7710 m/z.

The examples outlined in FIG. 5 demonstrate how different amidine scaffolds were able to withstand the strong acidic conditions (TFA) associated with both release of the peptide from the resin and cleavage of the side-chain protecting groups. The sequences also demonstrate how amidine formation was possible at different positions and between different residues along the backbone. Peptides were isolated in yields of 2-13% based on the reported loading of amino groups on the resin (Table 4). Isolated yields of the peptides are often low due to the linear nature of SPPS, and further complicated by the 3-phase sequence of (1) the problematic coupling of the thioamide residue, (2) conversion of thioamide into thioimidate, and (3) installation of the amidine that are not standard steps during SPPS. This procedure was not optimized for each peptide example (e.g., taking into account the sequence position). Instead, a standard procedure was applied for all peptides.

TABLE 4
Synthesis of biologically-relevant peptides.

| | Isolated | | Oxo-peptide |
|---|---|---|---|
| Peptide Sequence | Yield | Number Steps | Steps |
| Leu-Enkephlin (9) | 10% | 15 | 13 |
| Stonin (10) | 13% | 24 | 22 |
| Angiotensin (1-7) | 8% | 19 | 17 |
| (11a) | | | |
| Angiotensin (1-7) | 5% | 19 | 17 |
| (11b) | | | |
| Rubisolin-5 (12a) | 4% | 15 | 13 |
| Rubisolin-5 (12b) | 5% | 15 | 13 |
| C-peptide (13a) | 3% | 17 | 15 |
| C-peptide (13b) | 3% | 17 | 15 |

The results of Table 3 indicate that (3) installation of the amidine was efficient, or at least serviceable, to provide appropriate quantities of peptide for further study. Significant depression in isolated yield likely derives from the (1) coupling of the thioamide residue to the growing peptide. The thioamide coupling step was not quantitative, requiring Ac₂O capping in all cases to truncate unreacted chains. Additionally, inclusion of significant quantities of oxoamide can occur at these sites despite pure thioacyl reagent. Thus, we are currently working on strategies to streamline the 3 non-standard SPPS steps discussed above to improve the overall efficiency of the process.

The synthetic strategy developed in this study represents the first robust and general procedure for the introduction of amidines into the peptide backbone. Because amidines have remained largely unexplored in peptide science, this work is significant because it describes a generally applicable path to access unexplored peptide designs and architectures, enabled by the unique physicochemical properties and structure of the amidine (FIG. 1A). The approach centers around exploiting the site-selective insertion of thioamides as a reactive handle for conversion into amidine, but avoids the detrimental side-reactivity associated with previous methods that ultimately prevents successful activation of thioamides with stoichiometric amounts of precious transition metals. Namely, we convert the thioamide into a thioimidate that not only protects the integrity of the α-C stercochemistry of the thioamide residue during normal peptide synthesis, but also provides an electrophilic site for downstream conversion into an amidine. Notably, the amidine is introduced while the peptide is still protected and on-resin, thereby preventing any potential reactivity with side-chain functional groups. Thus, this work increases the utility and efficiency of exploiting thioimidates for the solid-phase peptide synthesis of thiopeptides by introducing a whole new avenue for peptide diversification, as amidinopeptides. We anticipate the exploration of this new chemistry within the context of novel peptide scaffolds and therapeutic compounds.

### Example 6. Cyclization of Amidines to Form (4H)-Imidazolones

### Introduction

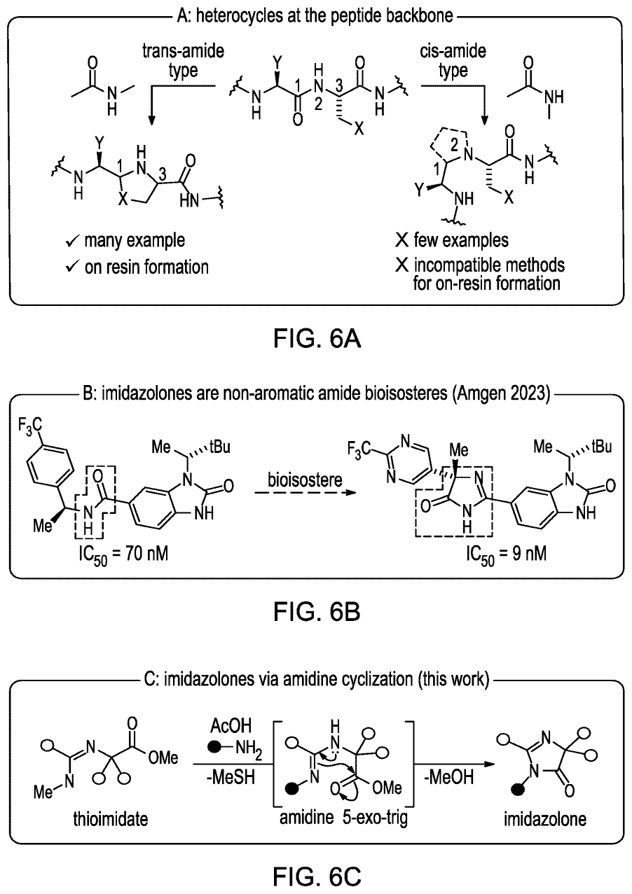

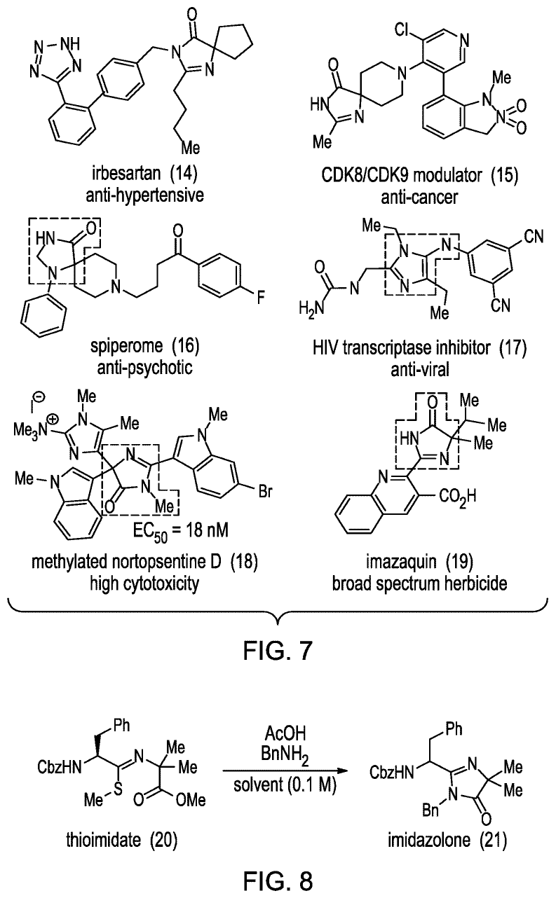

Here we report a method to form (4H)-imidazolone heterocycles within the peptide backbone. Imidazolones have unexplored effects on the conformational landscape of peptides, as well as unknown biological properties. Indeed, imidazolones have recently been shown to be a non-aromatic bioisostere of the amide bond with favorable pharmacological properties, creating a compelling need for further exploration (FIG. 6B). Additionally, (4H)-Imidazolones are high-value heterocycles appreciated for their anti-hypertensive, anti-cancer, anti-psychotic, anti-viral, and cytotoxic effects; they are also known to the agrochemical field as potent broad-spectrum herbicides (FIGS. 7, 14-19). Thus, new methods to form imidazolones under gentle conditions will enable access to amino-acid derived imidazolone natural products that have yet to be synthesized.

The new method reported here provides the first access to highly-functionalized and peptide-based (4H)-imidazolones with α-C chiral groups. Further, these imidazolones are substituted to give an all-cis-amide conformation. Unlike other cis-amide locked heterocycles, we can access imidazolone precursors in 2-3 steps from commercially-available building blocks, and install them on-resin at the N- and C-termini of the peptide, the middle of the peptide, and on the side-chain (enabling branched structures) (FIG. 6C). Finally, we show that our imidazolone performs better than other cis-amide surrogates in the pre-organization of a head-to-tail macrocyclization of natural product Mahafacyclin B. Notably, we do not observe any diketopiperazine side-products which have been observed quantitatively from the installation of other cis-amide bond surrogates.

### Results and Discussion

### Variation of Conditions of Imidazolone Formation.

We sought to explore various conditions to form imidazolone products in hopes that we could install imidazolone moicties during the course of SPPS. The Cbz-protected thioimidate dipeptide (20) was selected as a model substrate. The α,α-substitution of 2-aminoisobutyric acid (Aib) was chosen to mimic the majority of natural products and industrially-relevant imidazolones (FIG. 7) which contain α,α-substitution. Mono-substition leads to rapid racemization of the stereocenter at this position due to the thermodynamically-stable hydroxyimidazole tautomerization.

Table 5 illustrates various conditions used for imidazolone cyclization of thioimidate (20) to imidazolone (21) as shown in FIG. 8. Initial testing with our previous conditions for the formation of amidines from thioimidates yielded moderate yields (Table 5, entry 1). Unfortunately, 10 equivalents of the amine nucleophile complicates purification and restricts the scale of this chemistry. Therefore, in an effort to reduce the equivalents of amine required for this transformation, we employed Design of Experiments (DoE) methodology to locate optimal reaction conditions. DoE (compared to standard one-variable at a time optimization) enables us to understand not only the effects of our variables (amine and acid stoichiometries) on yield, but also how these variables affect each other. From this DoE optimization we observed two major trends: (1) amidine formation is driven primarily by the amount of amine nucleophile, which can be enhanced by concentration of the solution or stoichiometry, and (2) acid equivalency has a minor effect on yield (Table 5, entries 5-7), but can be important for the transformation.

TABLE 5
Conditions for imidazolone cyclization.

| | Eq. | Eq. | Solvent | Temp | Time | Yield |
|---|---|---|---|---|---|---|
| Entry | amine | AcOH | (v/v) | (° C.) | (h) | (%)^{a} |
| 1 | 10 | 1 | DMF:TFE | 23 | 24 | 73 |
| 2 | 5 | 2 | DMF:TFE | 23 | 24 | 93 |
| 3 | 5 | 0.5 | DMF:TFE | 23 | 24 | 50 |
| 4 | 3 | 1.25 | DMF:TFE | 23 | 24 | 64 |
| 5 | 1 | 2 | DMF:TFE | 23 | 24 | 31 |
| 6 | 1 | 0.5 | DMF:TFE | 23 | 24 | 25 |
| 7 | 1 | 5 | DMF:TFE | 23 | 24 | 27 |
| 8 | 3 | 1.25 | DMF | 23 | 24 | 7 |
| 9 | 3 | 1.25 | DCM:TFE | 23 | 24 | 71 |
| 10 | 3 | 1.25 | THF:TFE | 23 | 24 | 98 |
| 11 | 3 | 1.25 | MeCN:TFE | 23 | 24 | >99 |
| 12 | 1 | 0.5 | MeCN:TFE | 23 | 24 | 60 |
| 13 | 1 | 0.5 | MeCN:TFE | 70 | 8 | 90 |

^{a}Yield determined by crude NMR analysis with dimethyl terepthalate internal standard. Rows 2-6 were part of the full-factorial Design of Experiments (DOE) methodology.

We found that binary solvent mixtures containing 2,2,2-trifluroethanol (TFE) accelerate amidine installation which we attribute to solvent-enhanced hydrogen-bonding properties that may facilitate proton-transfer steps (Table 5, Entry 8). Solvent mixtures of THF: TFE and MeCN: TFE (1:1% v/v) were found to be conducive for this transformation, however DCM: TFE and DMF: TFE furnished imidazolone product in moderate yield (Table 5, Entries 7 & 9-11). Heating the reaction (Table 5, Entry 13) enabled us to form imidazolone products with stoichiometric amine quantities which is critical for the application of this chemistry in the context of SPPS where reaction rates are greatly reduced.

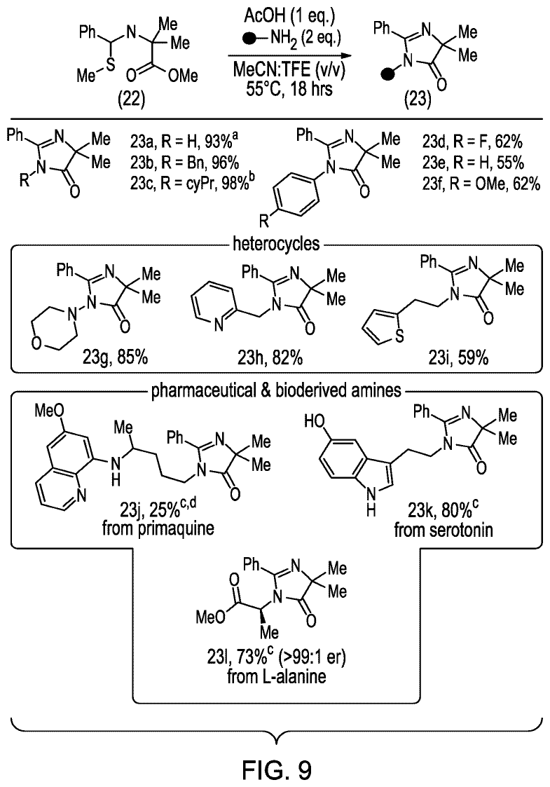

With the conditions giving the best results from Table 5, we explored the types of amine nucleophiles that were compatible with this reaction. FIG. 9 illustrates the scope of amine nucleophiles tolerated in imidazolone formation. Isolated yields shown. Concentration of all reactions was 0.25 M in 7. ^{a}No AcOH added. ^{b}40° C. ^{c}Ammonium salt neutralized with TEA (2 eq.) additive. ^{d}DMF:TFE (v/v) as solvent. ^{e}3 eq. of amine used. Ammonium salt neutralized with TEA (3 eq.) additive. We found that ammonium acetate (23a) and sterically-unencumbered primary amines (23b-23c) formed imidazolone products in very high yields. Anilines (23d-23f) produced the corresponding imidazolones in moderate yield, attributed to the decreased nucleophilicity of these amines due to delocalization of the nucleophilic lone-pair into the aromatic π-system. Imidazolone formation also tolerated an array of pharmaceutically-valuable heterocyclic functionalities including an α-nucleophile (23g), pyridines (23h and 23j), thiophene (23i) and indole (23k).

Pharmaceutical and bioderived amines (23j-231) with multiple functional groups were also well-tolerated. Diminished yield for primaquine derivative (23j) was attributed to the poor solubility observed for primaquine bisphosphate in organic solvents. Strong acid ammonium salts (i.e. HCl, H₃PO₄) were neutralized in-situ via addition of stoichiometric amount of triethylamine (TEA). We found that the strong acid salts alone were not able to catalyze the formation of imidazolones without addition of a weak acid. Amino acid nucleophiles were compatible with this chemistry and the reaction conditions did not racemize the stereogenic center of 231 despite the use of heat and acid. We found that more sterically-hindered amines or electron-deficient amines produced only trace imidazolone products which were not isolated.

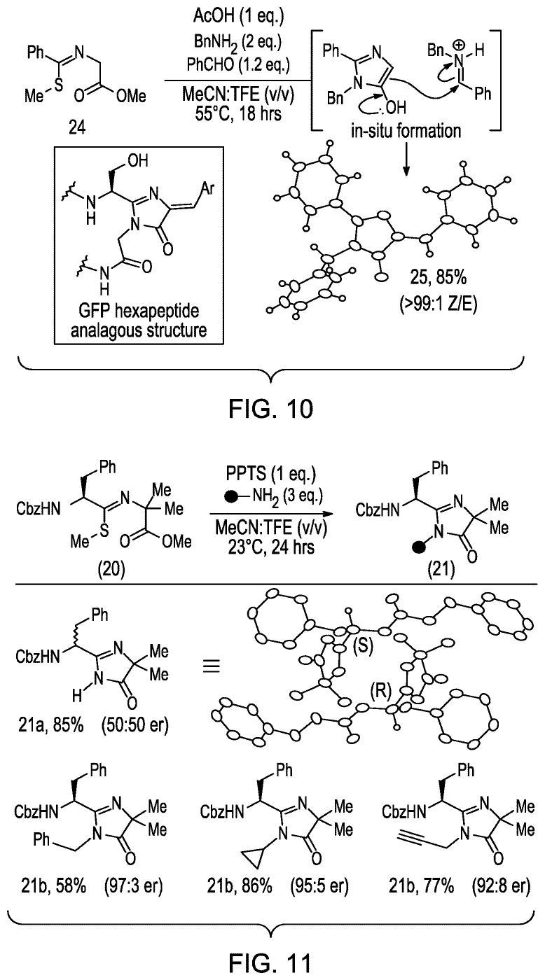

Originally reported as a trapping product, the benzylidene imidazolone adduct is recognized as the primary fluorophore in GFP. We therefore envisioned that in-situ imidazolone cyclization of 24, a glycine methyl ester thioimidate derivative, would enable us to form the benzylidene structure through an acid-catalyzed aldol reaction. A one-pot tandem cyclization-condensation with benzaldehyde afforded the benzylidene product 25 in high yield with excellent selectivity (FIG. 10).

### Stereoretentive Conditions for α-Chiral Imidazolones.

With an understanding of the scope of amine nucleophiles, we sought to explore the tolerance of our reaction on peptidic substrates. Notably, methods to generate chiral-enriched variants of imidazolones are largely underdeveloped. While our initial attempt to synthesize unsubstituted imidazolone 21a (FIG. 11) provided good yield, a subsequent crystal structure displayed a non-centrosymmetric space group, indicative of a mixture of both R- and S-imidazolones in the crystal (FIG. 11). Yields in FIG. 11 are isolated yields, and non-stereogenic H-atoms of 21a were omitted for clarity. The formation of thioimidates and amidines on peptidic substrates has been well-studied and the conditions employed do not racemize the α-position of peptides. Previously, Drabina and co-workers had reported trifluoroacetic acid (TFA) was capable of racemizing Boc-L-proline-imidazolone during the course of Boc-deprotection. Thus, we hypothesized that the reaction conditions led to the racemization of the α-stereochemistry after formation of the imidazolone, presumably through protonation of the imidazolone nitrogen, leading to an increase in acidity of the α-CH. We therefore sought conditions to minimize racemization of the final product.

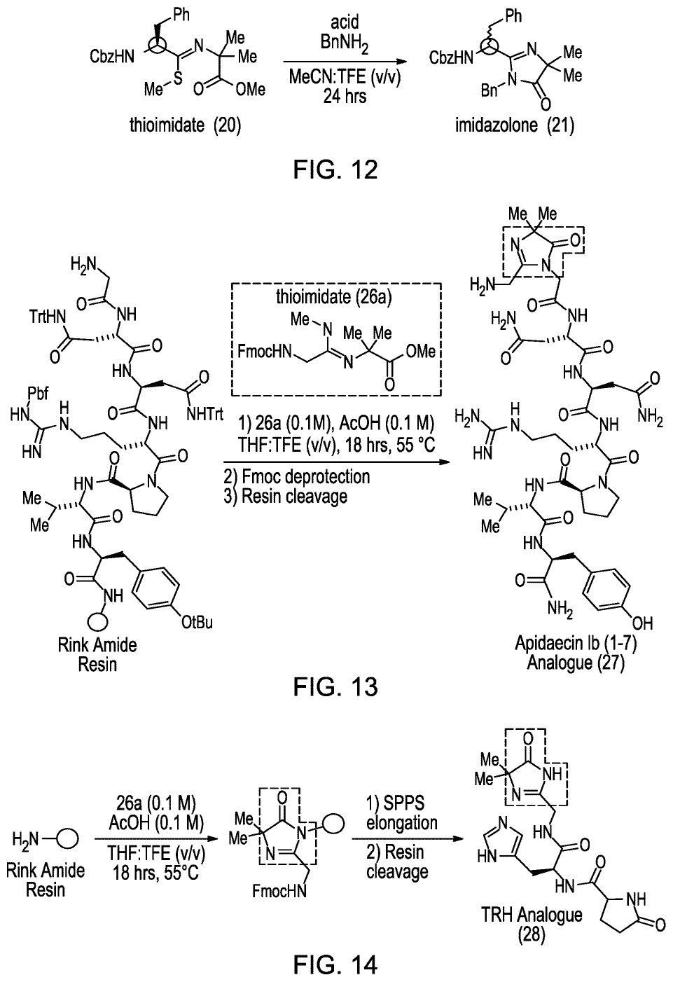

Table 6 illustrates conditions used for stereoretention of α-position of imidazolones in the cyclization of thioimidate (20) to imidazolone (21) as shown in FIG. 12. To identify if the pKₐ of the acid used for the transformation impacted the racemization, we explored pyridinium p-toluenesulfonate (PPTS, pKₐ=5.2) and hexafluoroisopropanol (HFIP, pKₐ=8.3) compared to acetic acid (AcOH, pKₐ=4.8). Intuitively, weaker acids such as PPTS and HFIP should protonate the imidazolone to a lesser extent and reduce racemization. Unfortunately, protonation of the thioimidate is also necessary for initial formation of the amidine ahead of cyclization (FIG. 6C). Accordingly, weak acids like HFIP did not furnish imidazolone in sufficient yield (Table 6, entry 2-4). We then attempted the reaction at lower temperatures, resulting in an enantiomeric ratio (er) of 90:10 using AcOH, however attempts to further improve the er using AcOH were unsuccessful (Table 6, entry 6 & 7). Thus, a combination of a weaker acid, PPTS, and room temperature reaction conditions provided the best compromise between isolated yield and enantiomeric ratio (Table 6, Entry 8).

TABLE 6
Conditions used for stereoretention
of the α-position of imidazolones.

| | Eq. | | Eq. | Temp | Isolated | |
|---|---|---|---|---|---|---|
| Entry | amine | Acid | acid | (° C.) | yield (%) | er |
| 1 | 1 | AcOH | 1 | 70 | 75 | 50:50 |
| 2 | 3 | AcOH | 1 | 50 | 89 | 77:23 |
| 3 | 3 | PPTS | 1 | 50 | 83 | 83:17 |
| 4 | 3 | HFIP | 1 | 50 | 34 | 87.5:12.5 |
| 5 | 3 | AcOH | 1 | 23 | 71 | 91:9 |
| 6^{a} | 3 | AcOH | 1 | 23 | 79 | 85.5:14.5 |
| 7 | 3 | AcOH | 0.5 | 23 | 40 | 94.5:5.5 |
| 8 | 3 | PPTS | 1 | 23 | 58 | 97:3 |
| 9 | 5 | PPTS | 1 | 23 | 71 | 73:27 |
| 10 | 10 | PPTS | 1 | 23 | 78 | 77:23 |

^{a}Reaction concentration 0.25M

With the determined conditions, we then prepared the first examples of enantiopure α-chiral imidazolones (21b-21d). We found that nucleophilic alkyl amines performed best for this chemistry to yield imidazolones in an overall >92:8 er. Our attempts to use aniline derivatives or hindered amines did not yield imidazolone products due to their poor room temperature reactivity. Interestingly, despite the development of stereoretentive conditions, attempts to generate enantiopure variants of unsubstituted imidazolone 21a were not successful.

### Solid-Phase Imidazolone Formation.

With an efficient method to form imidazolones established, we sought to incorporate these heterocycles into peptides through conventional solid-phase peptide synthesis (SPPS) procedures.

### N-Terminal Imidazolone Formation.

We selected Apidaecin Ib (1-7, H-GNNRPVY-NH₂) as our model peptide to test imidazolone cyclization using the free —NH₂ group on the resin as our amine nucleophile. Apidaecin Ib and synthetic derivatives are currently being evaluated for their potential to treat multidrug resistant gram-negative pathogens. Recently, Moore and co-workers showed that N-terminal guanidinylation of Apidaecin Ib peptide enhanced the anti-microbial activity and proteolytic stability. We hypothesized that imidazolones, a basic heterocyclic motif, might impart similar activity to the guanidinylated form by introducing a site for protonation. Positive charge has also been demonstrated as a useful tool to enhance the accumulation and uptake of anti-bacterial peptides and small molecules. However, on-resin installation of imidazolones required new considerations for our reaction conditions. THF was chosen as a co-solvent instead of MeCN because it swells polystyrene-based SPPS resins similarly to DMF and DCM, but provided the highest yields in our solution-phase solvent screening (Entries 4 & 9-11, Table 5). Since the amine nucleophile is immobilized on resin, we utilize an excess of thioimidate, Fmoc-Gly^{SMe}-Aib-OMe (26a, FIG. 13) for the reaction. We found that the typical concentration employed for amide coupling on resin (0.1 M) was sufficient to effect complete installation of the imidazolones when treated with 0.1 M 26a and 0.1 M AcOH under gentle heating conditions at 55 C (FIG. 13). Final resin cleavage and purification afforded 27, the N-terminal imidazolone analogue of Apidaccin Ib (1-7) (9% yield over all synthetic steps and HPLC purification).

### C-Terminal Imidazolone Formation.

To form C-terminal imidazolones we hypothesized that reaction of thioimidate with the amine of an amide resin itself (e.g., Rink amide) could yield N-unsubstituted imidazolones, enabling facile access to C-terminal imidazolone analogues in which the imidazolone is the direct link to the solid support. We selected C-terminal imidazolone 28 which mimics the molecular shape of the proline found at the C-terminus of the thyrotropin-releasing hormone (TRH). The clinically-used synthetic analogue of TRH, taltirelin, contains an unnatural dihydropyrimidine heterocycle. We envisioned that the imidazolone synthesis platform, which enables rapid diversification of the imidazolone core through SPPS, could be used in the drug discovery of new TRH analogues. After iterative cycles of SPPS coupling and Fmoc-deprotection the imidazolone remained intact and no discernible by-products related to scaffold degradation were isolated upon HPLC purification of peptide 28 (54% yield over all synthetic steps and HPLC purification). FIG. 14 illustrates imidazolone cyclization on Rink amide resin linker and subsequent clognation toward C-terminal imidazolone TRH analogue (28).

### Side-Chain Functionalization and Branched Peptides.

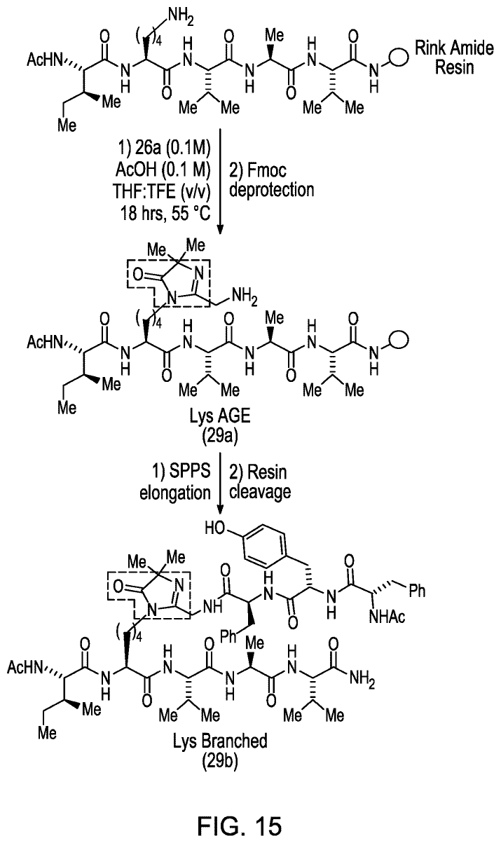

(4H)-Imidazolones are also a well-known class of biological metabolites known as advanced glycation end products (AGEs), which are non-enzymatic metabolites formed by the reaction of protein side-chains and sugars. AGEs are a product of normal metabolism, however high levels of AGEs have been linked to oxidative stress and inflammation; which are indicative of metabolic disorders, namely diabetes. AGEs react with the side-chains of cell surface receptor proteins, and alter their structure and function. For this reason we chose the peptide H-IKVAV-NH₂ which is a functional component of the laminin al chain, an extracellular matrix (ECM) protein responsible for cell adhesion and appreciated for its use as a hydrogel. By selectively deprotecting the ϵ-NH₂ of the Lys residue (using a commercially-available 4-methoxytrityl protecting group), we could install a side-chain imidazolone modification to form 29a which mimics AGE formation common to Arg and Lys side chains. Branched peptides have been appreciated by the medicinal chemistry community for their enhanced proteolytic stability. Accordingly, subsequent elongation from the side-chain imidazolone afforded a branched peptide structure 29b, another demonstration of the stability of the imidazolone core to SPPS conditions. FIG. 15 illustrates imidazolone formation on Lys side-chain to yield AGE-related modification (29a) and subsequent elongation to the Lys branched peptide (29b)

### Imidazolones With α-Stereochemistry.

With an effective method to install imidazolones directly on to solid-support, we sought to evaluate whether the stereochemistry of α-chiral imidazolones could withstand the basic (Fmoc-deprotection) and acidic (global deprotection and cleavage) conditions required for SPPS. We subjected a sample of 21c to 75% TFA in DCM (v/v) to mimic the conditions required for resin cleavage and global deprotection. To our delight, short exposure times of 1 hour showed <1% epimerization of the α-position.

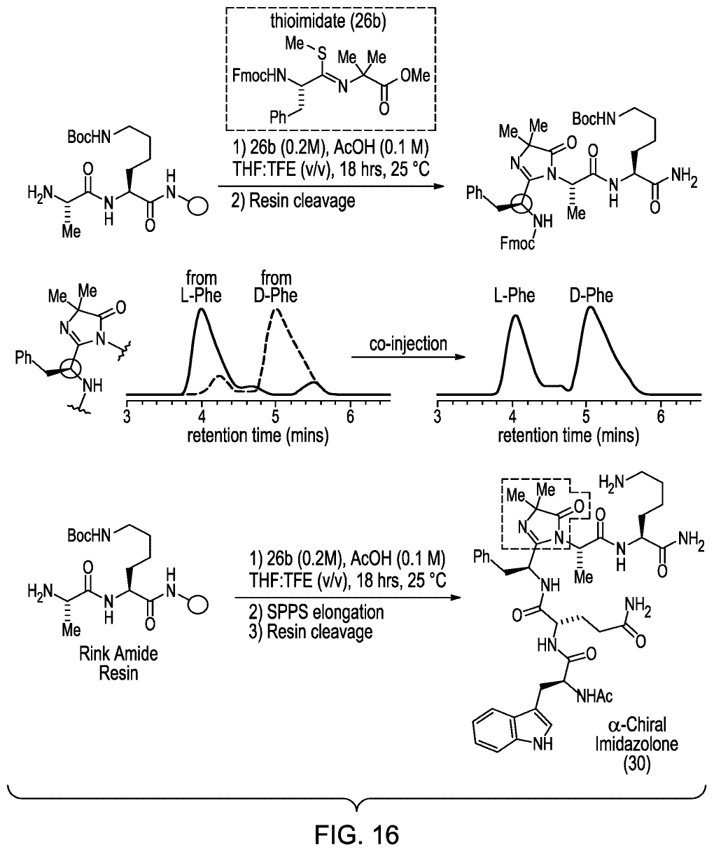

To evaluate the stability of the stereochemistry during the longer exposure times needed for on-resin installation of the imidazolone, we synthesized authentic L- and D-thioimidate precursors and reacted them with loaded resin (H-AK-NH₂) to quantify the degree of epimerization of the α-C stereochemistry during imidazolone installation. We found that our standard conditions of 0.1 M thioimidate and AcOH with heating led to 25% racemization of the stereochemistry. By increasing the concentration of the thioimidate dimer in solution to 0.2 M (with 0.1 M AcOH) we were able to effect installation at room temperature without the need for heating. Room temperature conditions led to <10% epimerization of the stereochemistry according to HPLC-MS which we validated by co-injection of the L- and DPhe variants (FIG. 16). Subsequent deprotection, elongation and cleavage led to α-chiral imidazolone (30) (7% over all synthetic steps and HPLC purification).

(cis)-Amide Bond Surrogates and Peptide Cyclization.

Finally, with effective conditions to install imidazolones on-resin, we sought to explore their utility as cis-amide bond surrogates. Head-to-tail macrocylization of peptides is assisted by the introduction of at least one cis-amide bond which helps to pre-organize the ends of cyclic peptides. Mahafacyclin B (cyclo-TFFGFFG), a cyclic peptide natural product appreciated for its anti-malarial properties, has been used as a benchmark structure to test head-to-tail cyclization strategies. Native cyclization of the linear Mahafacyclin B yields only 30% of the cyclic peptide and requires extremely long reaction times of up to 3 days. Jolliffe and coworkers, however, found that pseudoproline, a cis-amide surrogate derived from condensation with acetone at the Thr site, could pre-organize the peptide for cyclization. Their strategy yielded 50% of the target cyclic peptide in less than 3 hours, underscoring the importance of cis-amide linkages in assisting the cyclization of Mahafacyclin B. Robinson and coworkers used a non-canonical amino acid with alkene side chains and a ring-closing metathesis catalyst to form a tethered linkage to pre-organize the peptide. Their tethered structure yielded 60% of the cyclic peptide in 4 hours which could be later deprotected using ring-opening metathesis to yield a Mahafacyclin B analogue. We opted to use Mahafacyclin B as a benchmark cyclization for our cis-amide inducing imidazolone.

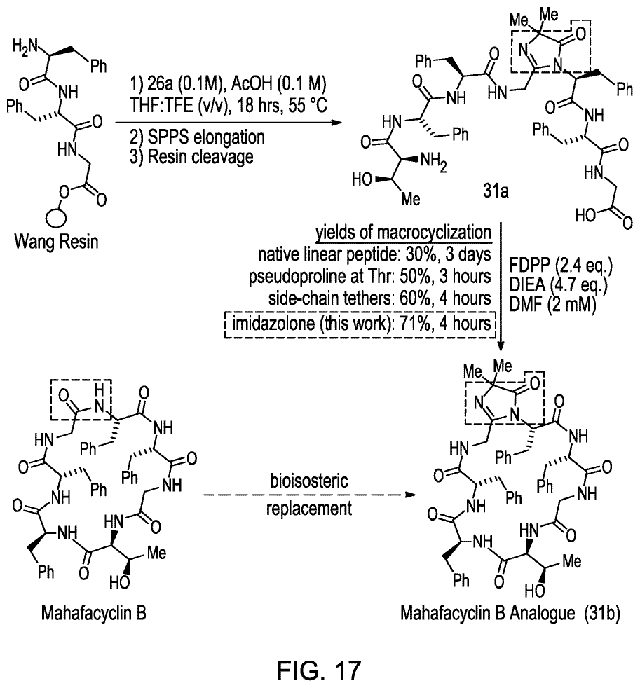

FIG. 17 illustrates the use of imidazolones as cis-amide surrogates to pre-organize head-to-tail macrocyclization of Mahafacyclin B analogue with bioisosteric replacement. To a Gly-loaded Wang resin we synthesized and installed our imidazolone at the central glycine residue. Cleavage from Wang resin afforded the linear peptide precursor (HTFFG^{Imi}FFG—OH, 31a, 12% yield over all synthetic steps and HPLC purification) which was allowed to react under the same macrocyclization conditions employed by Jollife and Robinson using a pentafluorophenyl diphenylphosphinate (FDPP) coupling reagent. Gratifyingly, the isolated yield of peptide 31b (71% yield over cyclization and HPLC purification) was higher than either previously reported macrocyclization of the Mahafacyclin B sequence. Additionally, since imidazolones have recently been employed as bioisosteric replacements for the amide bond, the geometric constraints imparted by imidazolones on bioactive linear peptides may be a useful strategy to retain bioactivity in cyclic form.

### Conclusions.

(4H)-imidazolones are important heterocycles found in natural products, pharmaceuticals, agrochemicals, fluorescent probes, and biological metabolites. Our method enables access to novel imidazolone scaffolds which can be synthesized rapidly from commercially available starting materials. We have shown that imidazolone cyclization is tolerated by a wide-scope of primary amine nucleophiles, with excellent functional group tolerance. Previous methods to form imidazolones required high temperatures and strong acid or base to form. The mild conditions employed enable us to access imidazolone products with α-stereochemistry—derived from amino acid starting materials—the first examples of stereochemical retention at this position.

Additionally, we show that imidazolones can be easily incorporated onto the N-terminus or side-chain of an elongating peptide chain during SPPS by reaction with a thioimidate dipeptide. C-terminal installation can be achieved by direct reaction with amide resins, which cleave to the free N H imidazolone. Imidazolones also tolerated peptide elongation after installation, enabling them to be installed in to the center of peptides and on to sidechains. Side-chain imidazolones provided access to branched peptides and AGE-related products with potential for the study of receptors for AGEs (RAGE). As a newly discovered bioisostere of the amide bond, our general method for insertion and functionalization of imidazolone motifs will assist in the discovery of other small-molecule and peptide-based therapeutics.

Finally, the geometric constraints imparted by the imidazolone's heterocycle leads to cis-amide surrogates which are especially useful in pre-organizing linear peptides for head-to-tail macrocyclization. We show that the imidazolone surrogate performs better than existing methods for the head-to-tail cyclization of the Mahafacyclin B sequence. We anticipate that this work will assist in the synthesis of currently inaccessible imidazolone natural products, assist in the design of cyclic peptides and peptide-based therapeutics, and provide a new conformational tool for peptide chemists to interrogate the effect of a cis-amide conformation on peptide interactions.

### Example 7. Hypothetical. Use of Leaving Group Atoms Other Than Oxygen

The synthesis of amidines described in the Examples above is expanded from reaction of a nitrogen-containing reagent with a thioimidate starting material to reaction of the nitrogen-containing reagent with a starting material compound including an imine that includes —X—R¹ bound to an imine carbon atom, as shown in Scheme 1.

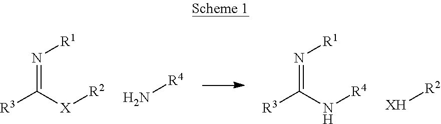

The same or similar reactions can be used as described in the Examples above. In Scheme 1, X can be —S—, —O—, or —NH—. The variable R¹ can be alkyl, aryl, H group, amino acid, or peptide in solution or bound to solid support. The variable R2 can be alkyl, aryl, or H group. The variable R³ can be alkyl, aryl, H group, amino acid, or peptide in solution or bound to solid support. The variable R⁴ can be alkyl, aryl, H group, amino acid, or peptide in solution or bound to solid support.

### Example 8. Solid-Supported Nitrogen-Containing Reagent

The synthesis of amidines described in the Examples above is expanded to the use of solid-supported nitrogen-containing reaction. Instead of a solid-bound thioimidate and an solution-phase amine reaction partner, a method is used that directly couples a solution-phase thioimidate reagent to the resin-bound peptide chain that displays the amine.

Reagents related to 6 in Scheme 2 were synthesized (3 steps from commercially available Fmoc-amino acids), wherein SS represents the solid support (resin). Reaction with the nitrogen-containing reagent as either the resin-bound N-terminus of the peptide (2) or ammonia equivalent produced substituted amidines depending on the identity of R. The variable X was —O—, —S—, or —NH—.

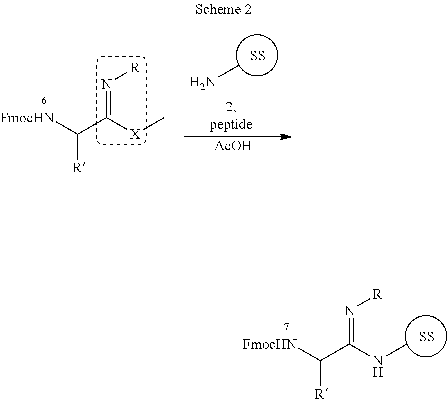

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects.

The following exemplary aspects are provided, the numbering of which is not to be construed as designating levels of importance:

Aspect 1 provides a method comprising:
reacting a starting material compound comprising an imine that comprises —X—R¹ bound to an imine carbon atom of the imine with a nitrogen-containing reagent to form a product compound comprising an amidine in place of the imine —X—R¹ in the starting material compound;
wherein
   X is —S—, —O—, or —NH—, and
   R¹ is substituted or unsubstituted (C₁-C₂₀)hydrocarbyl, wherein R¹ is optionally bonded to a solid support.

Aspect 2 provides the method of Aspect 1, wherein X is —O—.

Aspect 3 provides the method of Aspect 1, wherein X is —S—.

Aspect 4 provides the method of Aspect 1, wherein X is —NH—.

Aspect 5 provides the method of any one of Aspects 1-4, wherein R¹ is (C₁-C₂₀)alkyl.

Aspect 6 provides the method of any one of Aspects 1-5, wherein R¹ is methyl.

Aspect 7 provides the method of any one of Aspects 1-6, wherein R¹ is bonded to

the solid support.

Aspect 8 provide the method of any one of Aspects 1-7, wherein R¹ is free of bonds to a solid support.

Aspect 9 provides the method of any one of Aspects 1-8, wherein the imine is directly bonded to two carbon atoms in the starting material compound.

Aspect 10 provides the method of any one of Aspects 1-9, wherein the product compound is identical to the starting material compound other than the transformation of the imine —X—R¹ to the amidine.

Aspect 11 provides the method of any one of Aspects 1-10, wherein the amidine comprises ═NH, ═NR², —NH², —NHR², or —NR²₂, wherein at each occurrence R² is independently chosen from —OH, substituted or unsubstituted (C₁-C₂₀)hydrocarbyl, substituted or unsubstituted (C₁-C₂₀)alkyl, substituted or unsubstituted (C₆-C₂₀)aryl, and (C₆-C₂₀)heteroaryl.

Aspect 12 provides the method of any one of Aspects 1-11, wherein the amidine comprises —NH₂, —NHR₂, or —NR²₂, wherein at each occurrence R² is independently chosen from —OH, substituted or unsubstituted (C₁-C₂₀)hydrocarbyl, substituted or unsubstituted (C₁-C₂₀)alkyl, substituted or unsubstituted (C₆-C₂₀)aryl, and (C₆-C₂₀)heteroaryl.

Aspect 13 provides the method of any one of Aspects 1-12, wherein the amidine comprises ═NH or ═NR².

Aspect 14 provides the method of any one of Aspects 1-13, wherein the amidine comprises —NH₂.

Aspect 15 provides the method of any one of Aspects 1-14, wherein the amidine comprises —NHR² or —NR²₂, wherein R² is phenyl, benzyl, hydroxyl, or (C₁-C₂₀)alkyl.

Aspect 16 provides the method of any one of Aspects 1-15, wherein the amidine comprises —NHR², wherein R² is phenyl, benzyl, hydroxyl, or (C₁-C₂₀)alkyl.

Aspect 17 provides the method of any one of Aspects 1-16, wherein the amidine comprises —N—R²₂ wherein the two R² groups together are a substituted or unsubstituted (C₂-C₂₀)hydrocarbyl that forms a ring with the nitrogen atom, wherein the hydrocarbyl group is optionally interrupted with 0, 1, 2, or 3 groups chosen from —O— and —S—.

Aspect 18 provides the method of any one of Aspects 1-17, wherein the amidine comprises —NHR², wherein R² is 3-halophenyl, 3-hydroxyphenyl, 3-(C₁-C₁₀) alkoxy-phenyl, 1,4-teterahydroxyoxazinyl, thiophenyl(C₁-C₁₀)alkyl, indolyl(C₁-C₁₀)alkyl, or pyridinyl(C₁-C₁₀)alkyl.

Aspect 19 provides the method of any one of Aspects 1-18, wherein the reaction of the starting material compound with the nitrogen-containing reagent is performed in the absence of added solvent.

Aspect 20 provides the method of any one of Aspects 1-19, wherein the reaction of the starting material compound with the nitrogen-containing reagent is performed in an added solvent.

Aspect 21 provides the method of Aspect 20, wherein the added solvent comprises an organic solvent.

Aspect 22 provides the method of any one of Aspects 20-21, wherein the added solvent comprises a polar aprotic solvent.

Aspect 23 provides the method of any one of Aspects 20-22, wherein the added solvent comprises a halogenated solvent, chloroform, methylene chloride, tetrahydrofuran, dimethylformamide, 1,2-dimethyoxyethane, 1,3-dioxolane, dimethylsulfoxide, dimethyl acetamide, a fluoroalcohol, or a combination thereof.

Aspect 24 provides the method of any one of Aspects 20-23, wherein the added solvent comprises dimethylformamide, 2,2,2-trifluoroethanol, or a combination thereof.

Aspect 25 provides the method of any one of Aspects 20-24, wherein the added solvent comprises a volume ratio of dimethylformamide to 2,2,2-trifluoroethanol of 0.1:1 to 10:1.

Aspect 26 provides the method of any one of Aspects 20-25, wherein the added solvent comprises a volume ratio of dimethylformamide to 2,2,2-trifluoroethanol of 0.8:1 to 1.2:1.

Aspect 27 provides the method of any one of Aspects 20-26, wherein the added solvent is substantially free of water.

Aspect 28 provides the method of any one of Aspects 1-27, wherein a reaction milieu comprising the starting material compound and the nitrogen-containing reagent is substantially free of water.

Aspect 29 provides the method of Aspect 28, wherein the reaction milieu is 0 wt % to 2 wt % water.

Aspect 30 provides the method of any one of Aspects 28-29, wherein the reaction milieu is 0 wt % to 0.1 wt % water.

Aspect 31 provides the method of any one of Aspects 1-30, wherein a reaction milieu comprising the starting material compound and the nitrogen-containing reagent further comprises an acid.

Aspect 32 provides the method of Aspect 31, wherein the acid protonates the imine —X—R¹ in the starting material compound.

Aspect 33 provides the method of any one of Aspects 31-32, wherein the acid comprises a carboxylic acid, a mineral acid, an organic acid, formic acid, a sulfonic acid, or a combination thereof.

Aspect 34 provides the method of any one of Aspects 31-33, wherein the acid comprises acetic acid.

Aspect 35 provides the method of any one of Aspects 31-34, wherein compared to the amount of starting material compound present in the reaction milieu, 0.1 to 5 equivalents of the acid are present.

Aspect 36 provides the method of any one of Aspects 31-35, wherein compared to the amount of starting material compound present in the reaction milieu, 0.5 to 2 equivalents of the acid are present.

Aspect 37 provides the method of any one of Aspects 1-36, wherein the reacting of the starting material compound and the nitrogen-containing reagent is performed at room temperature.

Aspect 38 provides the method of any one of Aspects 1-37, wherein the reacting of the starting material compound and the nitrogen-containing reagent is performed at a temperature of 10° C. to 40° C.

Aspect 39 provides the method of any one of Aspects 1-38, wherein the reacting of the starting material compound and the nitrogen-containing reagent is performed at a temperature of 15° C. to 30° C.

Aspect 40 provides the method of any one of Aspects 1-39, wherein the reacting of the starting material compound and the nitrogen-containing reagent is performed at ambient pressure.

Aspect 41 provides the method of any one of Aspects 1-40, wherein the reacting of the starting material compound and the nitrogen-containing reagent is performed at a pressure of 20 kPa to 150 kPa.

Aspect 42 provides the method of any one of Aspects 1-41, wherein the reacting of the starting material compound and the nitrogen-containing reagent is performed at a pressure of 80 kPa to 120 kPa.

Aspect 43 provides the method of any one of Aspects 1-42, wherein the reacting of the starting material compound and the nitrogen-containing reagent is performed for a duration of 1 min to 72 h.

Aspect 44 provides the method of any one of Aspects 1-43, wherein the reacting of the starting material compound and the nitrogen-containing reagent is performed for a duration of 1 h to 48 h.

Aspect 45 provides the method of any one of Aspects 1-44, wherein the starting material compound and the product compound are peptides.

Aspect 46 provides the method of Aspect 45, wherein the peptides are dipeptides.

Aspect 47 provides the method of any one of Aspects 45-46, wherein the peptides are tripeptides.

Aspect 48 provides the method of any one of Aspects 45-47, wherein the peptides are polypeptides.

Aspect 49 provides the method of any one of Aspects 1-48, wherein the nitrogen-

containing reagent is part of the starting material compound, wherein formation of the amidine comprises an intramolecular ring-forming reaction.

Aspect 50 provides the method of Aspect 49, wherein the nitrogen-containing reagent comprises a primary amine or a secondary amine.

Aspect 51 provides the method of any one of Aspects 49-50, wherein the nitrogen-containing reagent comprises a secondary amine that is part of an aromatic or aliphatic ring.

Aspect 52 provides the method of any one of Aspects 1-51, wherein the nitrogen-containing reagent is a separate compound from the starting material compound.

Aspect 53 provides the method of Aspect 52, wherein compared to the amount of starting material compound present in the reaction milieu, 1 to 20 equivalents of the nitrogen-containing compound are present.

Aspect 54 provides the method of any one of Aspects 52-53, wherein compared to the amount of starting material compound present in the reaction milieu, 1 to 10 equivalents of the nitrogen-containing compound are present.

Aspect 55 provides the method of any one of Aspects 52-54, wherein the nitrogen-containing reagent comprises the primary amine.

Aspect 56 provides the method of Aspect 55, wherein the primary amine comprises aniline, benzylamine, hydroxylamine, or a combination thereof.

Aspect 57 provides the method of any one of Aspects 52-56, wherein the nitrogen-containing reagent comprises the secondary amine.

Aspect 58 provides the method of Aspect 57, wherein the secondary amine comprises piperidine, dimethylamine, or a combination thereof.

Aspect 59 provides the method of any one of Aspects 52-58, wherein the nitrogen-containing reagent comprises the ammonium salt.

Aspect 60 provides the method of Aspect 59, wherein the ammonium salt comprises an ammonium chloride, bromide, iodide, acetate, nitrate, bicarbonate, nitrite, cyanide, amide, cyanate, or hydroxide.

Aspect 61 provides the method of any one of Aspects 59-60, wherein the ammonium salt comprises ammonium chloride, ammonium acetate, or a combination thereof.

Aspect 62 provides the method of any one of Aspects 59-61, wherein a reaction milieu comprising the starting material compound and the nitrogen-containing reagent is substantially free of acids.

Aspect 63 provides the method of any one of Aspects 52-62, wherein the nitrogen-containing reagent comprises the structure R²-NH₂ or R²₂—NH, wherein at each occurrence R² is independently chosen from —OH, substituted or unsubstituted (C₁-C₂₀)hydrocarbyl, substituted or unsubstituted (C₁-C₂₀)alkyl, substituted or unsubstituted (C₆-C₂₀)aryl, and (C₆-C₂₀)heteroaryl.

Aspect 64 provides the method of any one of Aspects 52-63, wherein the amidine comprises ═NR², —NHR², or —NR²₂.

Aspect 65 provides the method of any one of Aspects 52-64, wherein the amidine comprises —NHR² or —NR²₂.

Aspect 66 provides the method of any one of Aspects 52-65, wherein the nitrogen-containing reagent comprises ammonium acetate, ammonium chloride, aniline, benzylamine, piperidine, dimethylamine, hydroxylamine, or a combination thereof.

Aspect 67 provides the method of any one of Aspects 1-66, wherein the method comprises performing the reacting of the starting material compound and the nitrogen-containing reagent while the starting material compound is bonded to a solid support.

Aspect 68 provides the method of Aspect 67, wherein the method further comprises bonding the starting material compound, or a precursor compound thereof, to the solid support.

Aspect 69 provides the method of Aspect 68, further comprising performing one or more synthetic transformations of the precursor compound while the precursor compound is bonded to the solid support to form the starting material compound bonded to the solid support.

Aspect 70 provides the method of any one of Aspects 67-69, further comprising cleaving the product compound from the solid support.

Aspect 71 provides the method of any one of Aspects 1-66, wherein the method comprises performing the reacting of the starting material compound and the nitrogen-containing reagent while the nitrogen-containing reagent is bonded to a solid support.

Aspect 72 provides the method of Aspect 71, wherein the method further comprises bonding the nitrogen-containing reagent, or a precursor compound thereof, to the solid support.

Aspect 73 provides the method of Aspect 71-72, further comprising performing one or more synthetic transformations of the precursor compound while the precursor compound is bonded to the solid support to form the nitrogen-containing reagent bonded to the solid support.

Aspect 74 provides the method of any one of Aspects 1-73, further comprising isolating and/or increasing the purity of the product compound.

Aspect 75 provides the method of any one of Aspects 1-74, further comprising subjecting the product compound to chromatography to increase the purity thereof.

Aspect 76 provides the method of any one of Aspects 1-75, further comprising reacting a precursor compound comprising an X-amide to form the starting material compound comprising the imine —X—R¹ in place of the X-amide in the precursor compound.

Aspect 77 provides the method of Aspect 76, wherein the starting material compound is identical to the precursor compound other than the transformation of the X-amide to the imine —X—R¹.

Aspect 78 provides the method of any one of Aspects 1-77, further comprising reacting a precursor compound comprising a thioamide to form the starting material compound comprising the imine —X—R¹ in place of the thioamide in the precursor compound.

Aspect 79 provides the method of Aspect 78, wherein the starting material compound is identical to the precursor compound other than the transformation of the thioamide to the thioimidate, carboximidate, or aminoimidate.

Aspect 80 provides the method of any one of Aspects 76-79, wherein reacting the precursor compound to form the starting material compound comprises reacting the precursor compound with an alkyl iodide in the presence of a base.

Aspect 81 provides the method of any one of Aspects 1-80, wherein a yield of the product compound from the starting material compound is 0.1% to 100%.

Aspect 82 provides the method of any one of Aspects 1-81, wherein a yield of the product compound from the starting material compound is 40% to 99%.

Aspect 83 provides the method of any one of Aspects 1-82, wherein a reaction milieu comprising the starting material compound and the nitrogen-containing reagent is substantially free of silver or silver-containing compounds.

Aspect 84 provides the method of any one of Aspects 1-83, further comprising further comprising reacting the amidine intramolecularly with a carbonyl carbon to form a cyclized product comprising a 4H-imidazolone and/or a derivative thereof.

Aspect 85 provides the method of Aspect 84, wherein the method comprises reacting the amidine with the carbonyl carbon to form the cyclized product in-situ in a same reaction container and/or reaction mixture used to react the starting material compound with the nitrogen-containing reagent to form the product compound.

Aspect 86 provides the method of any one of Aspects 84-85, wherein reaction conditions used to form the cyclized product are the same as reaction conditions used to form the product compound.

Aspect 87 provides the method of any one of Aspects 84-86, wherein the product compound comprises the structure

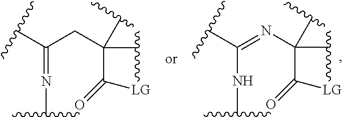

and wherein the cyclized product comprises the structure:

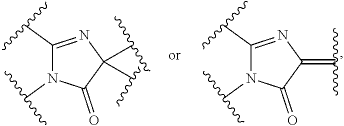 [SMILES: *C1=NC(*)(*)C(=O)N1* ; *C1=NC(=*)C(=O)N1*]

wherein LG is a leaving group.

Aspect 88 provides the method of any one of Aspects 84-87, wherein the product compound comprises R²—N^{A}═CR³—NH—CR⁴R⁵-C^{A}(O)—LG or R²—N^{A}H—CR³═N—CR⁴R⁵—C^{A}(O)—LG, wherein
R³ is chosen from —H, —OH, —OR⁶, substituted or unsubstituted (C₁-C₂₀)hydrocarbyl, substituted or unsubstituted (C₁-C₂₀)alkyl, substituted or unsubstituted (C₆-C₂₀)aryl, and substituted or unsubstituted (C₆-C₂₀)heteroaryl,
R⁴ and R⁵ are independently chosen from —H, —R⁶, —OH, —NH₂, —NHR⁶, —OR⁶, and —SR⁶,
at each occurrence R⁶ is independently chosen from substituted or unsubstituted (C₁-C₂₀)hydrocarbyl, substituted or unsubstituted (C₁-C₂₀)alkyl, substituted or unsubstituted (C₆-C₂₀)aryl, and substituted or unsubstituted (C₆-C₂₀)heteroaryl,
LG is a leaving group, and
reacting the amidine intramolecularly with the carbonyl carbon comprises reacting the nitrogen atom N^{A} with the carbonyl carbon C^{A}.

Aspect 89 provides the method of Aspect 88, wherein R³ is chosen from —H, phenyl, benzyl, hydroxyl, and (C₁-C₂₀)alkyl.

Aspect 90 provides the method of any one of Aspects 88-89, wherein R⁴ and R⁵ are independently chosen from —H, substituted or unsubstituted (C₁-C₂₀)alkyl, and substituted or unsubstituted (C₆-C₂₀)aryl.

Aspect 91 provides the method of any one of Aspects 88-90, wherein R⁴ and R⁵ are independently chosen from —H and (C₁-C₅)alkyl.

Aspect 92 provides the method of any one of Aspects 88-91, wherein R⁴ and R⁵ are —H.

Aspect 93 provides the method of any one of Aspects 88-92, wherein R⁴ and R⁵ are methyl.

Aspect 94 provides the method of any one of Aspects 88-93, wherein R⁶ is independently chosen from (C₁-C₂₀)hydrocarbyl, (C₁-C₂₀)alkyl, and (C₆-C₂₀)aryl.

Aspect 95 provides the method of any one of Aspects 88-94, wherein the leaving group is chosen from halide, —OR⁶, an O-ester, a suitable leaving group associated with peptide chemistry, a leaving group that promotes nucleophilic substitution, and —OCH₃.

Aspect 96 provides the method of any one of Aspects 88-95, wherein the leaving group is —OCH₃.

Aspect 97 provides the method of any one of Aspects 88-96, wherein the cyclized product has the structure:

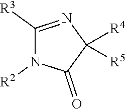

Aspect 98 provides the method of any one of Aspects 88-97, wherein the reaction of the amidine forms an intermediate having the structure:

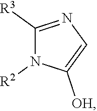

further comprising reacting the intermediate with an electrophile to form the cyclized product having the structure:

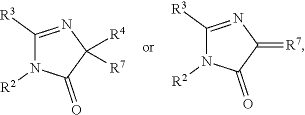

wherein R⁷ is a product of reaction of the electrophile with the intermediate.

Aspect 99 provides the method of Aspect 98, wherein the reaction of the intermediate with the electrophile is performed in-situ in a same reaction container and/or reaction mixture used to react the amidine intramolecularly with the carbonyl carbon.

Aspect 100 provides the method of any one of Aspects 84-99, comprising performing the reacting of the amidine intramolecularly with the carbonyl carbon under acidic conditions.

Aspect 101 provides the method of Aspect 100, wherein the acidic conditions comprise a reaction milieu that comprises an organic acid, a mineral acid, or a combination thereof.

Aspect 102 provides the method of Aspect 101, wherein the reaction milieu comprises acetic acid or pyridinium p-toluenesulfonate (PPTS).

Aspect 103 provides the method of any one of Aspects 84-102, wherein the 4H-imidazolone cyclized product comprises a cis-amide.

Aspect 104 provides the method of any one of Aspects 84-103, comprising reacting the product compound to form the cyclized product at a concentration of 0.1 M to 5 M in a reaction solvent.

Aspect 105 provides the method of any one of Aspects 84-104, comprising reacting the product compound to form the cyclized product at a concentration of 0.05 to 0.15 M.

Aspect 106 provides the method of any one of Aspects 84-105, wherein the product compound is reacted to form the cyclized product at a reaction temperature of 0° C. to 100° C.

Aspect 107 provides the method of any one of Aspects 84-106, wherein the product compound is reacted to form the cyclized product at a reaction temperature of 20° C. to 60° C.

Aspect 108 provides the method of any one of Aspects 106-107, further comprising maintaining the reaction temperature for a duration of 30 min to 48 h.

Aspect 109 provides the method of any one of Aspects 106-108, further comprising maintaining the reaction temperature for a duration of 10 h to 30 h.

Aspect 110 provides the method of any one of Aspects 84-109, comprising forming the cyclized product from the product compound at a yield of 20% to 100%.

Aspect 111 provides the method of any one of Aspects 84-110, comprising forming the cyclized product from the product compound at a yield of 60% to 100%.

Aspect 112 provides the method of any one of Aspects 84-111, comprising forming the cyclized product from the product compound at a conversion of the product compound of 50% to 100%.

Aspect 113 provides the method of any one of Aspects 84-112, comprising forming the cyclized product from the product compound at a conversion of the product compound of 80% to 100%.

Aspect 114 provides the method of any one of Aspects 84-113, further comprising performing the reacting of the product compound to form the cyclized product while the product compound is bonded to a solid support.

Aspect 115 provides the method of Aspect 114, further comprising cleaving the cyclized product from the solid support.

Aspect 116 provides a method comprising:
reacting a starting material compound comprising an imine that comprises —X—R¹ bound to an imine carbon atom of the imine with a nitrogen-containing reagent in a reaction milieu comprising a polar aprotic solvent and a fluoroalcohol to form a product compound comprising an amidine in place of the imine —X—R¹ in the starting material compound, wherein the nitrogen-containing reagent or the starting material compound is bonded to a solid support;
wherein
   the nitrogen-containing reagent comprises a primary amine, a secondary amine, an ammonium salt, or a combination thereof, and
   X is —S—, —O—, or —NH—, and
   R¹ is substituted or unsubstituted (C₁-C₂₀)hydrocarbyl, wherein R¹ is optionally bonded to the solid support.

Aspect 117 provides a method of forming a cyclized product, the method comprising:
intramolecularly reacting a compound comprising the structure

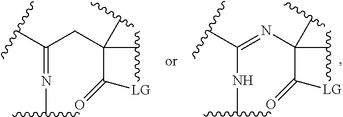

to form a cyclized product comprising the structure

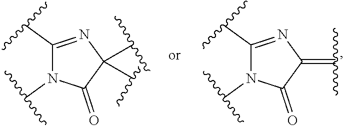 [SMILES: *C1=NC(*)(*)C(=O)N1* ; *C1=NC(=*)C(=O)N1*]

wherein LG is a leaving group.

Aspect 118 provides the method of any one or any combination of Aspects 1-117 optionally configured such that all elements or options recited are available to use or select from.

What is claimed is:

1. A method comprising:
reacting a starting material compound comprising an imine that comprises —X—R¹ bound to an imine carbon atom of the imine with a nitrogen-containing reagent to form a product compound comprising an amidine in place of the imine —X—R¹ in the starting material compound;
wherein
the nitrogen-containing reagent comprises a primary amine, a secondary amine, an ammonium salt, or a combination thereof,
X is —S—, —O—, or —NH—, and
R¹ is substituted or unsubstituted (C₁-C₂₀)hydrocarbyl, wherein R¹ is optionally bonded to a solid support.

2. The method of claim 1, wherein X is —O—.

3. The method of claim 1, wherein X is —S—.

4. The method of claim 1, wherein X is —NH—.

5. The method of claim 1, wherein the amidine comprises —NH₂, —NHR², ═NR², or —NR²₂, wherein at each occurrence R² is independently chosen from —OH, substituted or unsubstituted (C₁-C₂₀)hydrocarbyl, substituted or unsubstituted (C₁-C₂₀)alkyl, substituted or unsubstituted (C₆-C₂₀)aryl, and (C₆-C₂₀)heteroaryl.

6. The method of claim 1, wherein
the amidine comprises —NH₂, or
the amidine comprises —NHR² or —NR²₂, wherein R² is phenyl, benzyl, hydroxyl, or (C₁-C₂₀)alkyl, or
the amidine comprises —N—R²₂ wherein the two R² groups together are a substituted or unsubstituted (C₂-C₂₀)hydrocarbyl that forms a ring with the nitrogen atom, wherein the hydrocarbyl group is optionally interrupted with 0, 1, 2, or 3 groups chosen from —O— and —S—.

7. The method of claim 1, wherein the reaction of the starting material compound with the nitrogen-containing reagent is performed in an added solvent comprising an organic solvent, a polar aprotic solvent, a halogenated solvent, chloroform, methylene chloride, tetrahydrofuran, dimethylformamide, 1,2-dimethyoxyethane, 1,3-dioxolane, dimethylsulfoxide, dimethyl acetamide, a fluoroalcohol, or a combination thereof.

8. The method of claim 1, wherein a reaction milieu comprising the starting material compound and the nitrogen-containing reagent is 0 wt % to 2 wt % water.

9. The method of claim 1, wherein a reaction milieu comprising the starting material compound and the nitrogen-containing reagent further comprises an acid.

10. The method of claim 1, wherein the reacting of the starting material compound and the nitrogen-containing reagent is at a temperature of 10° C. to 40° C. and at a pressure of 20 kPa to 150 kPa.

11. The method of claim 1, wherein the starting material compound and the product compound are peptides.

12. The method of claim 1, wherein the nitrogen-containing reagent is part of the starting material compound, wherein formation of the amidine comprises an intramolecular ring-forming reaction.

13. The method of claim 1, wherein the nitrogen-containing reagent is a separate compound from the starting material compound.

14. The method of claim 13, wherein compared to the amount of starting material compound present in a reaction milieu including the starting material compound and the nitrogen-containing compound, 1 to 20 equivalents of the nitrogen-containing compound are present.

15. The method of claim 13, wherein the nitrogen-containing reagent comprises aniline, benzylamine, hydroxylamine, piperidine, dimethylamine, ammonium chloride, ammonium acetate, R²—NH₂, R²₂—NH, or a combination thereof, wherein at each occurrence R² is independently chosen from —OH, substituted or unsubstituted (C₁-C₂₀)hydrocarbyl, substituted or unsubstituted (C₁-C₂₀)alkyl, substituted or unsubstituted (C₆-C₂₀)aryl, and (C₆-C₂₀)heteroaryl.

16. The method of claim 1, wherein the method comprises performing the reacting of the starting material compound and the nitrogen-containing reagent while the nitrogen-containing reagent is bonded to the solid support, wherein R¹ is bonded to the solid support.

17. The method of claim 1, further comprising reacting a precursor compound comprising an X-amide to form the starting material compound comprising the imine —X—R¹ in place of the X-amide in the precursor compound.

18. The method of claim 1, wherein a reaction milieu comprising the starting material compound and the nitrogen-containing reagent is substantially free of silver or silver-containing compounds.

19. The method of claim 1, further comprising further comprising reacting the amidine intramolecularly with a carbonyl carbon to form a cyclized product comprising a 4H-imidazolone and/or a derivative thereof.

20. A method comprising:
reacting a starting material compound comprising an imine that comprises —X—R¹ bound to an imine carbon atom of the imine, wherein the starting material compound or the nitrogen-containing reagent is bonded to a solid support, with a nitrogen-containing reagent in a reaction milieu comprising a polar aprotic solvent and a fluoroalcohol to form a product compound comprising an amidine in place of the imine —X—R¹ in the starting material compound;
wherein
the nitrogen-containing reagent comprises a primary amine, a secondary amine, an ammonium salt, or a combination thereof, and
X is —S—, —O—, or —NH—, and
R¹ is substituted or unsubstituted (C₁-C₂₀) hydrocarbyl, wherein R¹ is optionally bonded to the solid support.
